SEQUENCE LISTING (3 sequences)

SEQ ID NO: 1 - DNA, 29 bp, Artificial Sequence
  Description of Artificial Sequence/note = synthetic construct
cataggtcgacctgtgtcagcatgtggga

SEQ ID NO: 2 - DNA, 29 bp, Artificial Sequence
  Description of Artificial Sequence/note = synthetic construct
cgtcggttggggactcacttcgaagatac

SEQ ID NO: 3 - DNA, 786 bp, Artificial Sequence
  Description of Artificial Sequence/note = synthetic construct
atgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtgggggtgattctgggggtccacttgtctgtaatggggtgcttcaaggtatcacatcatggggccctgagccatgtgccctgcctgaaaagcctgctgtgtacaccaaggtggtgcattaccggaagtggatcaaggacaccatcgcagccaacccctga

(19) United States
(12) Patent Application Publication
(10) Pub. No.: US 2004/0219163 A1
(43) Pub. Date: Nov. 4, 2004

(54) Human glandular kallikrein (hk2)-specific monoclonal antibodies that enhance or inhibit the enzymatic activity of hk2

(76) Inventors: John G. Frelinger, Pittsford, NY (US); Terrence L. Fisher, Rochester, NY (US); Mary Ann Nocera, Bergen, NY (US); Edith M. Lord, Rochester, NY (US)

Correspondence Address: NEEDLE & ROSENBERG, P.C., SUITE 1000, 999 PEACHTREE STREET, ATLANTA, GA, 30309-3915, US

(21) Appl. No.: 10/491,761

(22) PCT Filed: Oct. 3, 2002
(86) PCT No.: PCT/US02/31477

Related U.S. Application Data
(60) Provisional application No. 60/326,772, filed on Oct. 3, 2001.

(51) Int. Cl. A61K 39/12; A61K 39/21

(52) U.S. Cl. 424/186.1; 424/187.1; 424/188.1; 424/189.1

(57) ABSTRACT

Disclosed are compositions and methods for preferentially binding hK2 over PSA.

This application claims priority to U.S. Provisional Application No. 60/326,772 filed on Oct. 3, 2001, entitled “Human glandular kallikrein (hK2)-specific monoclonal antibodies that enhance or inhibit the enzymatic activity of hK2,” which application is herein incorporated by reference in its entirety.

### I. STATEMENT OF SUPPORT

This work was supported by USPHS grants CA28332 and CA70218, and by USPHS training grants T32AI07285 and T32CA09363. Thus, the government may have some rights in the invention.

### II. BACKGROUND

Human glandular kallikrein (hK2) and prostate-specific antigen (PSA) are serine proteases in the human kallikrein gene family that are 80% identical at the protein level. Like PSA, hK2 is expressed primarily in the prostate, making it an attractive bio-marker for prostate cancer development. In addition, its potent enzymatic activity can functionally affect the biology of prostate cancer

The human hK2 and PSA genes are members of a small multigene family called kallikreins (Diamandis E P, et al., Trends Endocrinol Metab 2000;11:54-60). Both PSA and hK2 show a highly restricted tissue distribution in men and are primarily, although not exclusively, expressed in epithelial cells of the prostate (Reviewed in (Rittenhouse H G, et al., Crit Rev Clin Lab Sci 1998;35:275-368)). The hK2 and PSA genes are highly homologous to one another and are 80% identical at the amino acid level. hK2 has tryptic-like activity whereas PSA is a chymyotryptic-like protease (Mikolajczyk S D et al., Eur J Biochem 1997;246:440-6). hK2 is autocatalytic, and can cleave PSA from the inactive proenzyme form into an enzymatically active form (Kumar A, et al., Cancer Res 1997;57:3111-4). The level of PSA in serum is one of the most widely used biomarkers for cancer (Oesterling J E., J Urol 1991;145:907-23) and recent reports have suggested hK2 levels in the serum can also be useful in prostate cancer diagnosis (Recker F, et al., Cancer 1998;83:2540-7; Saedi M S, et al., Clinical Chemistry 1998;44:2115-9; Kwiatkowski M K, et al., Urology 1998;52:360-5; Magklara A, et al., Clin Chem 1999;45:1960-6; Partin A W, et al., Urology 1999;54:839-45; Nam R K, et al., J Clin Oncol 2000;18:1036-42).

Expression of hK2 and PSA have been implicated in prostate cancer (Rittenhouse H G, et al., Crit Rev Clin Lab Sci 1998;35:275-368). For example, both PSA and hK2 can cleave insulin-like growth factor binding protein-3 (IGFBP-3) and thus would be expected to increase the bioavailability of IGF-1, a mitogenic factor for many cells that have been associated with increased risk of prostate cancer (Chan J M, et al., Science 1998;279:563-6). hK2 has also been implicated in the urokinase-type plasminogen activator cascade, suggested to be involved in the growth and invasiveness of many cancers including those of the prostate (Jarrard D F, et al., Invasion Metastasis 1995;15:34-45; Darson M E, et al., Urology 1997;49:857-62; Darson M F, et al., Urology 1999;53:939-44; Magklara A, et al., Urology 2000;56:527-32).

### III. SUMMARY

Disclosed are compositions and methods of using the compositions that involve hK2.

### IV. BRIEF DESCRIPTION OF THE DRAWINGS

FIG. 1 shows construction and analysis of hK2 transfected cell lines. (A) An hK2 cDNA was generated from LNCaP cells, characterized as described in the Material and Methods, and subcloned into the human β-actin expression vector into the Sal I and Hind III sites. FIG. 1(B) RT-PCR analysis of two independent clones of Line 1 cells transfected with the pHβ-hK2-neo expression vector. RT indicates the presence of reverse transcriptase in the reaction. No RT refers to the same reaction mix except that the reverse transcriptase enzyme was not added. These clones were analyzed with primers specific for mouse β-actin or for hK2. Bars to the left of the picture indicate the size of the expected product (hK2, 811 bp; β-actin, 596 bp).

FIG. 2 shows an analysis of antibody response in mice immunized with hK2 or PSA-expressing tumors. FIG. 2(A) BALB/c mice were immunized 4 times with irradiated Line 1/hK2 tumor cells and antibody titers determined. BALB/c.PSA transgenic mice were immunized similarly with either Line 1/PSA tumor cells FIG. 2(B), or with Line 1/hK2 tumor cells FIG. 2(C). The results of the serum analysis 10-14 days after the last immunization are shown. Antibodies reactive with hK2 (●) or PSA (▪) were measured. Serum from a mouse immunized with Line 1 (▴) was used as a negative control. Typical results from one mouse are shown for each immunization and all immunizations have been repeated 3× with similar results.

FIG. 3 shows an analysis of hybridomas generated from L1/hK2 immunized PSA transgenic mouse. Hybridoma supernatants were screened in an ELISA assay using a polyclonal rabbit anti-PSA antibody as the capture antibody and P815/hK2 supernatants or P815/PSA supernatants as the source of antigen. Hybridoma supernatants containing putative hK2 reactive monoclonals were screened as described in Materials and Methods, and reactivity of the hybridomas was detected using HRP-conjugated rabbit anti-mouse immunoglobulin. The reactivity patterns of 4 representative monoclonal antibodies (6B7, 3E6, IF8, and 2D3) are shown. Reactivity towards hK2 (●), PSA (□), or tissue culture media (Δ) was determined.

FIG. 4A shows anti-hK2 monoclonal antibodies do not react with PSA. hK2 A specific ELISA using 6B7 and 11C4 monoclonal antibodies was designed. Purified 6B7 was used as the capture antibody and the antigen was either supernatant from various transfected cell lines or purified PSA. Captured antigen was detected using the 11C4 monoclonal antibody followed by anti-mouse IgG3-HRP. The ELISA was developed using OPD and read at 490 nm. This ELISA was used with various antigen preparations: (●) P815-hK2 SN, (O) L1/PSA/IL2 SN, (Δ) Cortex PSA, (□) Calbiochem PSA, and PSA transgenic mouse prostate lysate containing PSA at a concentration of 300 μg/ml was used. This ELISA was been repeated 3× using similar samples with the same results. FIG. 4B shows an ELI-spot assay showing hK2-specificity of 1F8. The ELI-spot analysis was performed using the H117 monoclonal antibody that reacts with hK2 or PSA as the capture antibody. L1, L1/PSA, or L1/hK2 tumor cells were added and 3-fold serial dilutions were performed down the plate (2 dilutions are shown). Cell lines were grown in wells for 8 hours to secrete the antigen. The captured antigen was detected either using biotinylated 1F8 (top), which is hK2-specific or biotinylated H50 (bottom), which can react with both PSA and hK2. Antigen producing cells were visualized using AP-streptavidin and BCIP substrate. This ELI-spot assay has been repeated 2× with similar results.

FIG. 5 shows immunocytochemistry using the 6B7 monoclonal antibody. Paraffin embedded tissue sections were stained with 6B7 monoclonal antibody and visualized using horse anti-mouse IgG-biotin and streptavidin-HRP. Human prostate section stained with FIG. 5(A) 6B7 or FIG. 5(B) IgGI isotype control. Note the intense staining on the luminal side of the glands. To show prostate specificity, several different tissues FIG. 5(C-F) were stained on the same slide (“sausage block”) with 6B7; FIG. 5(C) prostate, FIG. 5(D) stomach, FIG. 5(E) muscle, FIG. 5(F) kidney. Bars indicate 50 microns. This staining was repeated 2× using various prostate sections with similar results.

FIG. 6 shows effects of hK2-specific monoclonal antibodies on function of hK2. hK2 purified from P815/hK2 SN was tested for trypsin-like enzymatic activity using the chromogenic substrate S-2302 and measuring the increase of absorbance at 405 nm (see Materials & Methods). FIG. 6(A) hK2-specific monoclonal antibodies were added to 50 ng hK2 at a 20 fold molar excess to determine their effect, if any, on hK2-activity. The increase in absorbance, which corresponds to an increase in hK2 activity, is shown versus time. The antibodies tested were 6B7(▴), 3C7 (●), and 1F8 (O). Mouse IgG1 (▪) and IgG2b (□) were included as negative controls and the absorbance @ 405 nm is minus background (no hK2), which was consistently <0.100, regardless of time. FIG. 6(B) The amount of hK2 was kept constant (25 ng) and substrate was used at a concentration of 0.5 mM, while the amount of antibody added was varied, effectively changing the molar ratio of antibody: hK2. This is expressed as percent activity of the hK2 when incubated without antibody, at a timepoint of 8 hours. Antibodies tested were same as in (A); 6B7(▴), 3C7 (●), 1F8 (O), and isotype controls IgG1(▪) and IgG2b (□) The mean values for the quintuplets of all antibodies are reported, with error bars depicting standard deviation, and similar assays with these antibodies were repeated 4× with similar results. Using the student's t-Test and comparing 3C7 and 1F8 with their appropriate isotype controls; * p=0.00000005 at 80:1 molar ratio and p<0.005 at all molar ratios>1.25:1, ** p=0.000018 at 80:1 molar ratio and p<.005 at all molar ratios>5:1.

FIG. 7 shows 3C7 can inhibit the activation of PSA by hK2. PSA was purified from either BALB/c.PSA transgenic mouse prostates FIG. 7(A) or L1/PSA/IL2 supernatant FIG. 7(B) and 2.5 ug PSA was incubated with 12.5 ug (a 1:1 molar ratio of Ab:PSA) of the mouse monoclonal antibodies 3C7 (E), RLS-DO6 FIG. 7(C), or mouse IgGI isotype control FIG. 7(G) for 1 hour on ice. The PSA-sensitive chromogenic substrate S-2586 (3-carbomethoxypropionyl-L-arginyl-L-prolyl-L-tyrosine-p-nitroanaline hydrochloride) was then added to the PSA/antibody mixture and monitored over time for an increase in absorbance at 405 nm, which is an indication of an increase in the enzymatic activity of PSA. In FIG. 7(C), 100 ng of purified, active hK2 was also added to the PSA and antibodies (a 25:1 molar ratio of Ab:hK2) and again followed for an increase in the enzymatic activity of PSA.

FIG. 8 shows tumor bearing mice that exhibit an increased percentage of B220⁺ cells and high levels of anti-Ova antibodies. Non-tumor draining FIG. 8(A) and tumor draining FIG. 8(B) LN were removed from BALB/c mice 20 days after tumor inoculation. Cell numbers were determined by trypan blue exclusion and found to be an average of 1.9±0.3×10⁶ cells total in the non-draining LN compared to 1.1±0.1×10⁷ cells in the tumor draining LN. Total LN cells were stained with a FITC-conjugated anti-B220 antibody or FITC-conjugated isotype matched control and subjected to flow cytometric analysis. The percentage of B220⁺ cells was determined by gating based on isotype control staining. The histograms shown are representative of 6 experiments performed. FIG. 8C) Sera from three individual mice injected with line 1/Ova (closed symbols) were collected at day 20 after tumor growth and assayed in an ELISA specific for anti-Ova antibodies. Sera from mice injected with parental line 1 cells were also collected and a representative sample is shown as a negative control (open symbols). An alkaline phosphatase-conjugated goat anti-mouse IgG reagent was used to detect total mouse IgG specific for Ova. A representative ELISA of 3 performed is shown. FIG. 8D) Tumor draining FIG. 8(B) and non tumor draining FIG. 8(B) LN cells were isolated from mice injected with line 1/Ova tumor cells for 21 days. Cells were plated in 96 well nitrocellulose microtiter plates coated with Ova protein. Cells secreting anti-Ova IgG were detected with alkaline phosphatase-conjugated goat anti-mouse IgG and spots were enumerated with a dissecting microscope. The graph shows the mean and standard error of three separate experiments.

FIG. 9 shows that Ova delivered by tumor cells induces anti-Ova antibodies of the IgG1 and IgG2a isotypes at a level similar to Ova in adjuvants. Three mice in each group were injected with 1×10⁵ viable line 1/Ova cells FIG. 9(B), 50 μg Ova in CFA FIG. 9(J) or 50 μg Ova in alum FIG. 9(H). Mice were bled at the indicated times and sera were tested in an Ova specific ELISA using alkaline phosphatase conjugated goat anti-mouse IgG1 FIG. 9(A) or alkaline phosphatase conjugated goat anti-mouse IgG2a FIG. 9(13) as the detecting antibody. The graphs show the endpoint titer of three individual mice at each time point tested.

FIG. 10 shows that antibody production in response to line 1 tumors is not dependent on IL-4. IL-4 deficient mice were injected with 1×10⁵ line 1/Ova tumor cells or 50 μg/ml Ova in CFA as a positive control. Mice were bled at day 18 and serum used in an anti-Ova specific ELISA for IgG1 (top panel) or IgG2a (bottom panel). BALB/c mice were also injected with line 1/Ova tumor cells for 18 days as a positive control for high anti-Ova IgG1 antibodies as shown previously (FIG. 9). Each point represents an individual mouse and the bars represent the geometric mean of all mice assayed. Statistically significant differences in anti-Ova IgG1 titers were observed between BALB/c mice and IL-4 deficient mice injected with line 1/Ova (p=0.02 by Mann-Whitney U test). By the same statistical criteria, IL-4 deficient mice injected with line 1/Ova tumors demonstrate significantly higher IgG2a titers than BALB/c mice injected with line 1/Ova (p=0.02).

FIG. 11 shows different forms of antigen delivered by H-2^{d} restricted tumors eliciting high antigen-specific antibody titers in BALB/c mice. BALB/c mice were injected with antigen transfected line 1 or EMT6 tumors (See Table 3). Tumors were allowed to grow for 16-21 days or until the mean leg diameter reached approximately 12-14 mm. Mice were bled and serum assayed in antigen specific ELISAs. Mouse antibodies were detected with HRP- or AP-conjugated goat anti-mouse IgG and ODs read at 490 or 405 nm respectively. Antibody titer was determined as the inverse dilution that gave an OD reading of twice background levels. This was done to normalize between the different ELISAs performed. Each point represents an individual mouse and the bar represents the geometric mean titer of all mice assayed. The standard error of the mean ranged between 980-2000 in mice injected with antigen expressing line 1 tumors and 360-14,000 in mice injected with antigen expressing EMT6 tumors.

FIG. 12 shows that Line 1, but not B16, tumors generate high antigen specific antibody titers in syngeneic and semi-allogeneic mice. BALB/c FIG. 12(A) or C57BL/6 FIG. 12(B) mice were injected with line 1/Ova FIG. 12(J) or line 1/PSA FIG. 12(F); B16/Ova FIG. 12(E) or B16/PSA FIG. 12(A) tumor cells. Filled symbols represent transfected line 1 cells whereas open symbols denote antigen expressing B16 tumors. (BALB/c×C57BL/6)F1 mice FIG. 12(C) were also injected with line 1/Ova FIG. 12(J), B16/Ova FIG. 12(E), line 1/PSA FIG. 12(F) or B16/PSA FIG. 12(A). After 16-21 days of tumor growth, mice were bled, sera collected and analyzed for antigen specific IgG using anti-Ova or anti-PSA specific ELISAs. Endpoint titers were determined by the inverse dilution that gave an OD reading of twice background levels. Each point represents an individual mouse and the bar depicts the geometric mean of all mice assayed. FIG. 12A is pooled data from 2 separate experiments. Similarly, the B16/Ova column in FIG. 12C is pooled data from 2 separate experiments. Antibody titers in F1 mice injected with line 1/Ova tumors were significantly higher than antibody titers of mice injected with B16/Ova tumors; p=0.008 by the Mann-Whitney U test. Similarly, antibody titers in mice injected with line 1/PSA were statistically different (p=0.02) than those titers of mice injected with B16/PSA.

FIG. 13 shows that Line 1 tumors act as adjuvants to induce anti-Ova antibodies in response to B16/Ova tumors. A) Line 1 tumors grow interspersed with B16/Ova tumors at the same rate as line 1 or B16/Ova tumors injected alone. Two×10³ line 1 cells were mixed with 2×10⁵ B16/Ova tumor cells and injected i. m. into (BALB/c×C57BL/6) F1 mice. After 18 days of tumor growth, mice were sacrificed and tumors excised for photography. FIG. 13B) B16/Ova tumors and B16/Ova mixed with line 1 maintain expression of Ova protein after 18 days of tumor growth in vivo. Tumor cells were injected as described above and following growth in vivo, tumors were excised, dissociated into single cell suspensions, lysed and assayed for levels of Ova protein by ELISA. The closed square represents B16/Ova tumor cells growing in vitro and the closed circle is a representative B16/Ova tumor grown in vivo. The open symbols represent 3 different line 1+B16/Ova tumors grown in vivo and the small circle represents a line 1 tumor grown in vivo. FIG. 13C) Mice injected as described above were bled at 18-21 days after tumor growth and serum analyzed for anti-Ova antibodies of the IgG isotype. Endpoint titers were determined by the inverse dilution that gave an OD reading of twice background levels. Each point represents an individual mouse and is pooled data from 2 separate experiments. The bar depicts the geometric mean of all mice assayed. Anti-Ova antibody titers in mice receiving a mixture of line 1 and B16/Ova tumors were significantly higher than titers in mice receiving an injection of B16/Ova alone (p=0.007 by the Mann-Whitney U test). FIG. 13D) B16 tumors do not suppress antibody synthesis induced by line 1/Ova tumors. Two×10⁵ B16 and 5×10⁴ line 1/Ova tumor cells were mixed and injected i. m. into (BALB/c×C57BL/6) F1 mice. Mice were bled at 18-21 days after tumor growth and serum analyzed for anti-Ova antibodies of the IgG isotype. Endpoint titers were determined by the inverse dilution that gave an OD reading of twice background levels. Each point represents an individual mouse and the bar depicts the geometric mean of all mice assayed.

FIG. 14 shows a vector used with hK2. Within the diagram, the *bp 1-4300 region is the 4.3 kb EcoR1-Alu1 fragment from the human beta-actin gene which is from isolate p14T beta-17 (Leavitt et. al. Mol. Cell. Biol. 1984, 4, 1961-1969). The promoter sequence can be found in Ng et. al. Mol. Cell. Biol. 1985, 5, 2720-2732. The cap site, 5′ untranslated region and IVS 1 positions are indicated above. There is no ATG codon present in the 5′ UT nor in the poly-linker region from the 3″ splice site to the BamH1 site. The region of bp 4300-4320 is in part derived from pSP64 poly-linker (Melton et. al. Nucl. Acids Res. 1984. 12, 7035-7056). The region *bp 4320-6600 is derived from pcDY1 (Okayama & Berg. Mol. Cell. Biol. 1983, 3, 280-289) and contains the pBR322 Amp resistance gene and bacterial origin plus the SV40 late region polyadenylation signal. The region *bp660-10000 is the Pvu11-EcoR1 fragment from pSV1-neo (Southern & Berg, J. Mol. App. Genet. 1982, 1, 327-341) containing the bacterial neo gene linked to the SV40 ori plus early promoter. Direction of transcription is as indicated.

### V. DETAILED DESCRIPTION

Before the present compounds, compositions, articles, devices, and/or methods are disclosed and described, it is to be understood that they are not limited to specific synthetic methods, specific recombinant biotechnology methods unless otherwise specified, or to particular reagents unless otherwise specified, as such may, of course, vary.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Abbreviations: Complete Freund's adjuvant, CFA; dendritic cells, DC; green fluorescent protein, GFP; lymph nodes, LN; ovalbumin, Ova; prostate specific antigen, PSA

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood. For example, if the value “10” is disclosed the “less than or equal to 10” as well as “greater than or equal to 10” is also disclosed.

“Optional” or “optionally” means that the subsequently described event or circumstance may or may not occur, and that the description includes instances where said event or circumstance occurs and instances where it does not. “Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art, which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

“Adjuvant” is anything, such as a molecule or mixture of molecules, capable of enhancing an immune response to an antigen.

Throughout this application, various publications are referenced. The disclosures of these publications in their entireties are hereby incorporated by reference into this application in order to more fully explain the disclosed compositions and methods. The references disclosed are also individually and specifically incorporated by reference herein for the material contained in them that is discussed in the sentence in which the reference is relied upon.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective permutation of these compounds may not be explicitly disclosed, each is specifically contemplated and described herein. For example, if a particular hK2 specific antibody is disclosed and discussed and a number of modifications that can be made to a number of molecules including the hK2 monoclonal antibody are discussed, specifically contemplated is each and every combination and permutation of the hK2 monoclonal antibody and the modifications that are possible unless specifically indicated to the contrary. Thus, if a class of molecules A, B, and C are disclosed as well as a class of molecules D, E, and F and an example of a combination molecule, A-D is disclosed, then even if it each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

B. Compositions

Disclosed are compositions and methods that relate to hK2 and PSA. Antibodies are often used in assays to show the presence or absence of the antigen for the antibody in the sampled assay. Often times, antibodies will cross react with more than one antigen because the recognition site for the antibody is wholly or partially present in more than one protein, such as closely related proteins. hK2 PSA are closely related proteins and it is desirable to have compositions that specifically recognize one or the other protein, without recognizing the other. Disclosed are compositions that bind hK2 specifically, relative to PSA. The disclosed compositions bind hK2 and show reduced cross reactivity with PSA. In addition, disclosed are compositions that enhance the activity of hK2. Also disclosed are compositions that inhibit the activity of hK2.

In certain embodiments the compositions can be antibodies, such as monoclonal or polyclonal antibodies, that bind hK2 specifically relative to PSA. On certain embodiments the antibodies enhance hK2 activity and in others the antibodies inhibit the hK2 activity.

In Example 1, a panel of antibodies was generated that represented a number of different isotypes (IgG1, IgG3, IgG2b).

The disclosed compositions can be used in the diagnosis of cancers, such as prostate cancer. hK2 levels in serum can be of diagnostic value for cancer detection, either alone or in conjunction with PSA (Kwiatkowski M K, et al., Urology 1998;52:360-5; Partin A W, et al., Urology 1999;54:839-45; Nam R K, et al., J Clin Oncol 2000;18:1036-42). Recent work has shown that the amount of hK2 helps determine whether or not the cancer is organ-confined (Haese A, et al., J Urol 2000;163:1491-7; Recker F, et al., Urology 2000;55:481-5). Immunohistochemical (Darson M F, et al., Urology 1997;49:857-62; Darson M E, et al., Urology 1999;53:939-44) and RT-PCR (Slawin K M, et al., Cancer Res 2000;60:7142-8) techniques indicate that the amount of hK2 protein and mRNA, respectively, correlates positively with cancer grade and progression. See also (Magklara A, et al., Urology 2000;56:527-32). A large, diverse panel of antibodies recognizing various epitopes of hK2 can be used in multi-part diagnostic assays, since the various forms and complexes of hK2 that are present in serum and tissues may preferentially express different epitopes under varying physiologic conditions.

Thus disclosed are assays using the compositions to identify the presence of hK2. It is also understood that these assays can be used in conjunction with other assays for the detection or diagnosis of cancers, such as prostate cancer. For example, the presence of PSA is a common marker for diagnosing prostate cancer, and the disclosed compositions and assays can be used in conjunction with any PSA assay, to further provide a diagnosis, based on the presence of the PSA and hK2.

The disclosed compositions can be used with any type of assay, such as an Elisa or an Elispot, or radioimmunoassays or chemiluminenesence assays.

The disclosed assays, such as the ELIspot assay used in Example 1, to examine the specificity of the antibodies can also be useful to monitor the expression of hK2 in metastatic cells. A series of studies have shown that PSA and hK2 mRNA could be detected using RT-PCR techniques (Corey E, et al., Urology 1997;50:184-8) and that prostate cancer cells were present in the bone marrow of many cancer patients (Melchior S W, et al., Clin Cancer Res 1997;3 :249-56) (Lange P H, Vessella R L., Cancer Metastasis Rev 1998;17:331-6). Such studies, by necessity, only looked at the mRNA levels, and could not determine the number of such hK2 expressing cells. Also, it is possible that amounts of hK2 mRNA may not correspond directly with the amount of hK2 protein, since the amount of hK2 mRNA is about 10 to 60% of PSA mRNA in prostate tissue, while the expression of hK2 protein is often 100 times less than PSA protein in seminal plasma or serum (Rittenhouse H G, et al., Crit Rev Clin Lab Sci 1998;35:275-368).

The disclosed compositions and methods allow for the examination and enumeration of individual tumor cells. In this regard, the ability to monitor expression at the single cell level has been useful for the analysis of negative variants in cancer vaccine therapies.

The presence of hK2 is indicated in cancers, such as prostate cancer. hK2 can cleave and inactivate insulin-like growth factor binding protein 3 (IGFBP-3) (Rittenhouse H G, et al., Crit Rev Clin Lab Sci 1998;35:275-368), thereby increasing the availability of insulin-like growth factor-1 (IGF-1), a mitogen for prostate epithelial cells (Cohen P, et al., Horm Metab Res 1994;26:81-4) whose levels have been correlated in epidemiological studies with increased prostate cancer risk (Chan J M, et al., Science 1998;279:563-6). hK2 can also affect the uPA pathway that is involved in the invasiveness of many tumor cells, including prostate (Jarrard D F, et al., Invasion Metastasis 1995;15:34-45), by activating single-chain urokinase-type plasminogen activator (uPA) (Frenette G, et al., Int J Cancer 1997;71:897-9), and by inactivating plasminogen activator inhibitor- (Mikolajczyk S D, Int J Cancer 1999;81 :438-42), an inhibitor of uPA. Such pleiotropic effects are reminiscent of the findings in the matrix metalloproteinase (MMP) system (reviewed by McCawley (McCawley L J, Matrisian L M., Mol Med Today 2000;6:149-56)) in which the varied roles of these enzymes in several steps of tumor growth and progression has led to the development and testing of MMP inhibitors in clinical trials (Drummond A H, et al., Ann N Y Acad Sci 1999;878:228-35). Analogously, since hK2 can act enzymatically on a number of other proteins, including uPA, IGF-binding proteins, and PSA, hK2 is can be a target for modulation.

A panel of hK2-specific, non-PSA cross-reactive monoclonal antibodies was produced using a tumor-immunization strategy. Human hK2 cDNA was transfected into a BALB/c tumor cell line and used to immunize PSA-expressing BALB/c.PSA transgenic mice. A B cell fusion was performed using spleen cells from a mouse immunized in this fashion. A panel of monoclonal antibodies was produced and shown to be hK2-specific using hK2-specific sandwich ELISA and ELIspot assays disclosed herein. One of the monoclonals (6B7) was used to detect hK2 in human prostate by immunohistochemistry. Interestingly, at least two of the antibodies affected the function of hK2. The 1F8 antibody enhanced the enzymatic activity of hK2 whereas the 3C7 antibody inhibited its function. These hK2-specific antibodies illustrate an approach disclosed herein for constructing B-cell hybridomas and provide useful reagents to examine the role of hK2 in the biology and detection of prostate cancer.

1. Compositions that Bind hK2

Disclosed are compositions that bind hK2. Also disclosed are compositions that preferentially bind hK2 over PSA.

In certain embodiments, the disclosed compositions preferentially bind hK2 over PSA with a cross reactivity of less than or equal to about 0.1%, about 0.01%, about 0.001%, about 0.0001%, about 0.00001%, or about 0.000001%. For example, it certain embodiments the binding to hK2 can be detected at about 0.0003 ng/ml, about 0.003 ng/ml, about 0.03 ng/ml, about 0.3 ng/ml, about 3 ng/ml, about 30 ng/ml, about 300 ng/ml, or about 3,000 ng/ml of composition, while no detection of binding to PSA can be detected at for example, about 3,000 ng/ml, about 30,000 ng/ml, about 300,000 ng/ml, about 3,000,000 ng/ml, about 30,000,0000 ng/ml.

In certain embodiments, there is a at least a 100 fold, 1,000 fold, 10,000 fold, 100,000 fold, 1,000,000 fold, 10,000,000 fold, or 100,000,000 fold difference between the Kd of the compositions and hK2 and the Kd between the compositions and PSA.

It is understood that the compositions can be anything which is capable of specifically binding hK2 over PSA as disclosed herein, including macromolecules, such as, antibodies, peptides, peptide memetics, functional nucleic acids, or small molecules.

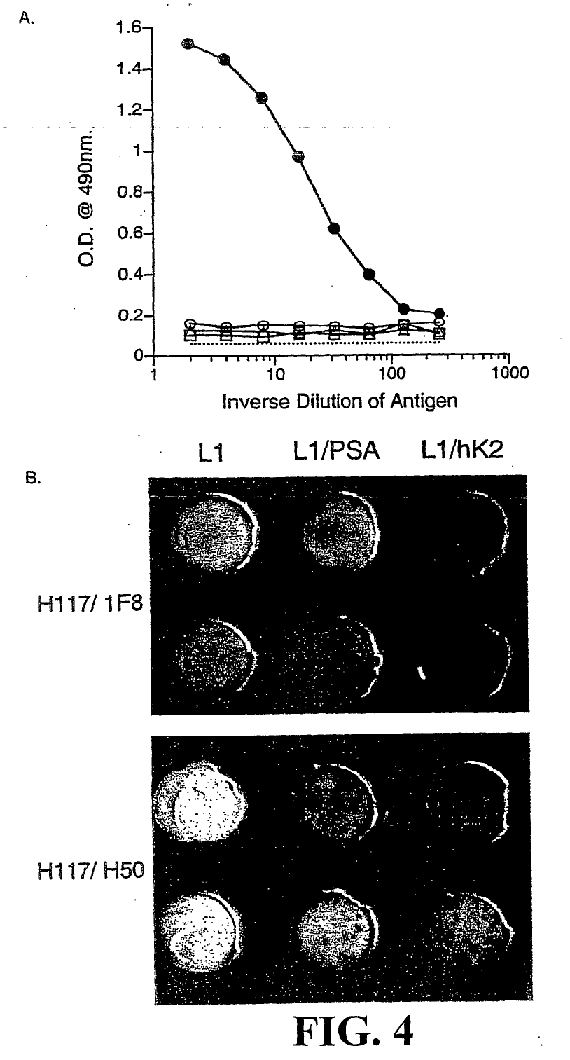

Cross reactivity can be determined, for example, using a disclosed sandwich ELISA using any two compositions. For example, a Elisa sandwich assay is disclosed that used two of the hK2-reactive monoclonal antibodies, 6B7 as the capture antibody and 11C4 as the detecting antibody. For example, in this type of assay P815/hK2 SN at a concentration of approximately 200 ng/ml is positive and is detectable to approximately 3 ng/ml. Several different sources of PSA were used in this assay and none showed reactivity. For example, purified PSA obtained from Calbiochem or Cortex at 50 ng/ml and recombinant PSA from the tumor cell line SN (L1/PSA/IL-2) at 700 ng/ml was negative. In order to determine if the antibodies would react with PSA at even higher concentrations, a sample of homogenized prostate from a transgenic mouse that contained PSA at a concentration of 300 ug/ml was tested and was completely negative by this assay (dotted line in FIG. 4), illustrating that this assay does not react with PSA even at extremely high amounts of antigen. Thus, together with the estimate that the 6B7/11C4 pair can detect hK2 at a concentration of approximately 3 ng/ml, the PSA cross-reactivity of this assay is less than 3/300,000 or 0.001%.

a) Antibodies that Bind hK2

The disclosed compositions can be antibodies that specifically recognize hK2 over PSA, as described herein, for example, monoclonal antibodies. In certain embodiments the disclosed compositions are monoclonal antibodies, in other embodiments the compositions can be polyclonal antibodies.

It is understood that those of skill in the art understand that the disclosed compositions, for example, specific antibodies to hK2, for example, monoclonal antibodies, are typically polypeptides which are encoded by nucleic acid. Those of skill in the art understand how to obtain and use these nucleic acids which are disclosed herein.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain typically has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each typically comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as scFv, sFv, F(ab′)2, Fab′, Fab and the like, including hybrid fragments. Thus, fragments of the antibodies that retain the ability to bind their specific antigens are provided. For example, fragments of antibodies which maintain hK2 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference at least for material related to antibodies and antigen binding proteins.

Optionally, the antibodies are generated in other species and “humanized” for administration in humans. Humanized forms of non-human (e.g., murine) antibodies are chimeric immunoglobulins, immunoglobulin chains or fragments thereof (such as scFv, sFv, Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

Disclosed are hybidoma cells that produces monoclonal antibodies. The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

Monoclonal antibodies may be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises HK2. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of HK2 expressed as a fusion protein with human IgG1 is injected into the host animal according to methods known in the art (e.g., Kilpatrick KE, et al. Gene gun delivered DNA-based immunizations mediate rapid production of murine monoclonal antibodies to the Flt-3 receptor. Hybridoma. 1998 December;17(6):569-76; Kilpatrick KE et al. High-affinity monoclonal antibodies to PED/PEA-15 generated using 5 microg of DNA. Hybridoma. 2000 August;19(4):297-302, which are incorporated herein by referenced in full for the the methods of antibody production) and as described in the examples.

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing domains of hK2 antibody as fission proteins. The antigen is produced by inserting a gene fragment in-frame between the signal sequence and the mature protein domain of the hK2 antibody nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against HK2. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA), or chemiluminescence assays. Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells, using the appropriate vectors described herein. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Optionally, such a non-immunoglobulin polypeptide is substituted for the constant domains of an antibody or substituted for the variable domains of one antigen-combining site of an antibody to create a chimeric bivalent antibody comprising one antigen-combining site having specificity for hK2 and another antigen-combining site having specificity for a different antigen.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W. H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J.Biol.Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with hK2. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment, which are discussed in greater detail below. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

Also provided is an antibody reagent kit comprising containers of the monoclonal antibody or fragment thereof and one or more reagents for detecting binding of the antibody or fragment thereof to the hK2. The reagents can include, for example, fluorescent tags, enzymatic tags, or other tags. The reagents can also include secondary or tertiary antibodies or reagents for enzymatic reactions, wherein the enzymatic reactions produce a product that can be visualized.

b) Non-Antibody compositions that Bind hK2

Also disclosed are compositions that preferentially bind hK2 over PSA that are non-antibody molecules, for example nucleic acids or peptides or peptide mimetics. In general, it is understood that nucleic acids can be made up of any naturally occurring nucleotide, such as guanosine, as well as any nucleotide derivative such as 7-deaza-guanosine or even for example, PNA where appropriate. Likewise, peptides, where appropriate, can be made up of any naturally occurring amino acid such as alanine, or amino acid derivatives.

(1) Functional Nucleic Acids

Functional nucleic acids are nucleic acid molecules that have a specific function, such as binding a target molecule, such as hK2 or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, such as hK2, or the functional nucleic acid molecules can possess a de novo activity independent of any other molecules. Functional nucleic acids can be made from any known nucleic acid or nucleic acid that is appropriate, for example, to increase in vivo stability or resistance to nucleases.

Functional nucleic acid molecules can interact with any macromolecule, such as DNA, RNA, polypeptides, or carbohydrate chains. Thus, functional nucleic acids can interact with mRNA that produces an antibody to hK2 or the genomic DNA of the same or they can interact with the polypeptide hK2 itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d})less than 10⁻⁶. It is more preferred that antisense molecules bind with a k_{d} less than 10⁻⁸. It is also more preferred that the antisense molecules bind the target molecule with a k_{d} less than 10⁻¹⁰. It is also preferred that the antisense molecules bind the target molecule with a k_{d} less than 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶. It is more preferred that the aptamers bind the target molecule with a k_{d} less than 10⁻⁸. It is also more preferred that the aptamers bind the target molecule with a k_{d} less than 10⁻¹⁰. It is also preferred that the aptamers bind the target molecule with a k_{d} less than 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). In the following discussion regarding dissociation constants, it is preferred that the target molecule comprise hK2 and the background binding molecule be PSA. It is preferred that the aptamer have a k_{d} with the target molecule at least 10 fold lower than the k_{d} with a background binding molecule. It is more preferred that the aptamer have a k_{d} with the target molecule at least 100 fold lower than the k_{d} with a background binding molecule. It is more preferred that the aptamer have a k_{d} with the target molecule at least 1000 fold lower than the k_{d} with a background binding molecule. It is preferred that the aptamer have a k_{d} with the target molecule at least 10000 fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Also disclosed are ribozymes, (for example, U.S. Pat. Nos. 5,334,711, 5,646,031, 5,595,873, 5,580,967) triplex forming functional nucleic acid molecules (U.S. Pat. No. 5,176,996) External guide sequences (EGSs) (WO 92/03566 by Yale, and Forster and Altman, Science 238:407-409 (1990) 5,168,053).

2. Compositions that Alter the Function of hK2

Also disclosed are compositions that can alter the functional properties of the hK2 protein. For example, disclosed are compositions which can enhance the enzymatic activity of the hK2. Also disclosed are compositions that can inhibit the enzymatic function of hK2. As discussed herein, these compositions can be any compositions that perform these particular functions. For example, the compositions can be antibodies, as disclosed herein including monoclonal antibodies. The compositions can also be non-antibody compositions as disclosed herein, such as aptamers or other peptides or peptide mimetics.

Also disclosed are compositions, wherein the molecule preferentially inhibits the enzymatic activity of hK2 over PSA.

Disclosed are compositions that can modulate PSA enzymatic activity by modulating the enzymatic activity of hK2. Also disclosed are compositions that can inhibit the enzymatic activity of PSA by inhibiting the enzymatic activity of hK2. Also disclosed are compositions that can enhance the enzymatic activity of PSA by enhancing the enzymatic activity of hK2.

It is preferred that the compositions affect a particular level of enzymatic activity as disclosed herein. The examples use an assay discussed in Mikolajczyk et al. (Milcolajczyk S D, Int J Cancer 1999;81 :438-42). This assay uses the chromogenic substrate S-2302 to analyze the enzymatic activity of hK2. The assay can be performed as follows. The antigen, such as hK2, can be purified; for example, over an antibody affinity column, and the purified fractions pooled (Wofsy L, Burr B., J Immunol 1969;103:380-2.). The enzymatic activity can be determined using a chromogenic substrate, such as H-D-prolyl-L-phenylalanyl-L-arginine-p-nitroaniline-dihydrochloride (S-2302, Chromogenix, Diapharma Group Inc, Westchester, OH), for hK2 (Mikolajczyk S D, et al., Eur J Biochem 1997;246:440-6). The chromogenic assay can be performed, for example, as follows. The purified antigen, such as hK2, can be incubated with 1M S-2302 in 0.1 M Tris-HCl, pH 7.8, 0.1 M NaCl in a final volume of 150 ul that contained 0.1% bovine serum albumin. The reactions can be performed in U-bottom microtiter plates at 37° C. The release of p-nitroanilide was followed at 405 nm using a Dynascan MRX ELISA reader. When antibodies are included in the assay, the purified antibody and antigen, such as hK2, can be incubated for 30 minutes at 37° C. prior to the addition of S-2302. Purified antibodies can be buffer exchanged in PBS before use. The is how the assay was performed in the Examples.

Disclosed are compositions that modulate the activity of hK2 by at least 1, 2, 4, 5, 10, 15, 20, 25, 30, 35, 40, 45, 50, 60, 70, 80, 90, 100, 150, 200, 250, 300, 400, 500, 750, 1000, 2000, or 5000%. Also disclosed are compositions that inhibit the activity of hK2 by at least 1, 2, 4, 5, 10, 15, 20, 25, 30, 35, 40, 45, 50, 60, 70, 80, 90, 100, 150, 200, 250, 300, 400, 500, 750, 1000, 2000, or 5000% as well as compositions that enhance the activity of hK2 by at least 1, 2, 4, 5, 10, 15, 20, 25, 30, 35, 40, 45, 50, 60, 70, 80, 90, 100, 150, 200, 250, 300, 400, 500, 750, 1000, 2000, or 5000%.

3. Compositions Identified by Screening with Disclosed Compositions/Combinatorial Chemistry

a) Combinatorial Chemistry

The disclosed compositions, for example, antibodies, can be used as targets or reagents for any combinatorial technique to identify molecules or macromolecular molecules that interact with the disclosed compositions in a desired way. The nucleic acids, peptides, and related molecules disclosed herein can be used as targets for the combinatorial approaches. The disclosed compositions can also be used within various combinatorial methods or screening procedures to identify molecules having a similar or improved property. Also disclosed are the compositions that are identified through combinatorial techniques or screening techniques in which the compositions or portions thereof, are used as the target or a reagent in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function, such as hK2, or preferentially bind the target molecule, such as hK2. The molecules identified and isolated when using the disclosed compositions are also disclosed. Thus, the products produced using the combinatorial or screening approaches that involve the disclosed compositions are also herein disclosed.

The disclosed compositions, such as monoclonal antibodies that bind hK2 specifically, or enhance hK2 activity or inhibit hK2 activity can be used to identify any other molecule having the same or similar activity. For example, the molecules can be bound to hK2, and those molecules that are competitively removed with the any of the disclosed compositions are desired because these molecules will have properties similar to molecule that they were competitively removed with.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A.,et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example a desired portin of hK2 is attached to a DNA binding domain of a transcriptional activation protein, such as Gal 4. By performing the Two-hybrid technique on this type of system, molecules that bind a particular portion of hK2, for example, can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2, 3-dihydrothiazoles (U.S. Pat. No. 5,847,150), quinolines (U.S. Pat. No. 5,840,500), PNA (U.S. Pat. No. 5,831,014), containing tags (U.S. Pat. No.5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

Screening molecules similar to the disclosed antibodies for preferential binding of hK2 over PSA or inhibition of hK2 enzymatic activity are disclosed. For example, the disclosed compositions can be used to isolate molecules, using the methods disclosed herein, that have similar recognition properties as the disclosed compositions, for example the disclosed monoclonal antibodies to compete it off. The disclosed compositions can be used in the disclosed methods, as for example, competitive binders. For example, a population of molecules can be incubated with hK2, molecules which are not bound can be washed away, and the remaining molecules can be incubated with the disclosed compositions, for example, the monoclonal antibodies, which have the desired properties. These compositions will compete with the molecules that recognize the hK2 in a similar way and by collecting the molecules obtained after this competition reaction will produce a population enriched for binding activity that is similar to the molecule which was used isolating the desired compounds. As used herein combinatorial methods and libraries included traditional screening methods and libraries as well as methods and libraries used in iterative processes.

b) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation of the target molecule's function. The molecules identified and isolated when using the disclosed compositions, such as, monoclonal antibodies, are also disclosed. Thus, the products produced using the molecular modeling approaches that involve the disclosed compositions, such as, monoclonal antibodies, are also herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossman, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

4. Compositions that Enhance Immune Responses to Antigens

As discussed herein, disclosed are compositions that function as an adjuvant for generating immune responses to an antigen. Also disclosed are compositions that function as an adjuvant for enhancing existing immune responses to an antigen.

When generating an immune response, antigens are injected into an animal, such as a mouse. The antigen is recognized as foreign, and an immune response develops, typically culminating in generating a panel of antibodies that bind the antigen. This response involves the activation of many different cell types and many different cell signaling cascades. It is understood that this response can be enhanced or generated by addition of a variety of materials. These materials are typically called adjuvants, and are often added with the antigen to the animal to enhance or in some cases generate the immune response, which will result in higher titers of the derised antibodies. One such adjuvant is called “Complete Freunds Adjuvant” (CFA). CFA can be added with an antigen and will enhance or stimulate the immune response to the antigen.

Disclosed are compositions and methods that function as adjuvants. It is disclosed that when an antigen is expressed in a cell, and that cell is grown in an animal an enhanced immune response is achieved. By enhanced, is meant that the amount of antibody titer for the antigen is increased relative to production of antibody to the antigen alone.

This adjuvant effect is seen with a variety of tumor cell lines, such as L1, EMT6, a breast cancer cell line, CT-26, a colon cancer cell line. A CT 26 line can be found in Griswold D P. And Corbett T H. (Griswold D P. And Corbett T H., Cancer. 36(6 Suppl):2441-4, (1975). Which is herein incorporated by reference at least for material related to colon cancer cell lines, such as CT-26).

The adjuvant effect also occurs whether the antigen is secreted, whether the antigen is bound to the surface of the cell, or whether the antigen is internal to the cell.

In certain embodiments the disclosed compositions have an adjuvant effect that causes an immune response that is at least 1, 2, 4, 5, 10, 15, 20, 25, 30, 35, 40, 45, 50, 60, 70, 80, 90, 100, 150, 200, 250, 300, 400, 500, 750, 1000, 2000, or 5000% greater than the immune response achieved when the compositions are not present. Also disclosed are compositions that have an adjuvant effect that causes an immune response that is at least 1, 2, 4, 5, 10, 15, 20, 25, 30, 35, 40, 45, 50, 60, 70, 80, 90, 100, 150, 200, 250, 300, 400, 500, 750, 1000, 2000, or 5000% of the adjuvant effect of CFA, ie disclosed are compositions that have an adjuvant effect that is less than the adjuvant effect of CFA, but that still have an adjuvant effect, as well as compositions that have an adjuvant effect greater than the adjuvant effect of CFA.

The adjuvant effect of the disclosed compositions can be determined as disclosed in Example 2.

Typically the disclosed compositions comprise a cell that expresses the antigen. The cells can be cell lines, such as tumor cell lines, such as an L1, EMT6, a breast cancer cell line, or CT-26, a colon cancer cell line. The antigens can be expressed using any of the disclosed expression systems, simply requiring that the antigen is expressed.

It is also understood that the compositions, such as the disclosed cellular adjuvants can be used in conjunction with traditional antibody generation techniques. For example, the disclosed cells can be added to an animal in combination with an antigen that is not explicitly expressed within the cell but rather is added exogenously to the organism. Thus, the cellular adjuvant can be used in combination with antigen administration, like CFA or other adjuvants would be used. Thus disclosed are compositions that comprises a cellular adjuvant and an antigen. The antigen can be either expressed by the cellular adjuvant or complexed with the cellular adjuvant. In addition the antigen and cellular adjuvant can be maintained and delivered to the organism in separate vehicles.

It is understood that the antigen can be any antigen. For example, the antigen can be hK2, but the antigen can be molecule or composition that will generate an immune response in a particular organism, such as a mammal, such as a mouse or bovine or ovine or human.

Disclosed are compositions, wherein the antigen comprises a viral antigen. For example the antigen can be an antigen that includes a peptide or protein or other molecule, such as nucleic acid or sugar, derived from a virus. Derived from means that the molecule is a molecule found as part of the virus or typically considered as part of the virus. Disclosed are viral antigens from any virus, such as Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

Disclosed are compositions, wherein the antigen comprises a bacterial antigen. The antigen can also be an antigen that includes a peptide or protein or other molecule, such as nucleic acid or sugar, derived from a bacteria. Derived from means that the molecule is a molecule found as part of the bacteria or typically considered as part of the bacteria. Disclosed are bacterial antigens derived from i Mycobacterium, Nocardia, Legionella, Salmonella, Shigella, Yersinia, Pasteurella, Actinobacillus, Listeria, Brucella, Cowdria, Chlamydia, Coxiella, Rickettsial, Ehrlichia, Staphylococcus, Streptococcus, Bacillus, Escherichia, Vibrio, Campylobacter, Neiserria, Pseudomonas, Haemophilus, Clostridium, Yersinia.

For example, disclosed are baterial antigens derived from M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachoinatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, or other Yersinia species.

Disclosed are compositions, wherein the antigen comprises a parasite antigen. The antigen can also be an antigen that includes a peptide or protein or other molecule, such as nucleic acid or sugar, derived from a parasite. Derived from means that the molecule is a molecule found as part of the parasite or typically considered as part of the parasite. Disclosed are parasite antigens derived Toxoplasma, Plasmodium, Trypanosoma, Leishmania, Schistosoma, or Entamoeba. Also disclosed are parasite antigens derived from Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica.

It is also understood that the antigens can include molecules that are unique to other types of compositions, such as cells, and which can generate an immune response in a particular organism. For example, certain types of cells, such as cancer cells, express different proteins than the analogous non-cancer cell, or they express a mutated form of a protein. It is understood that the antigen can be a peptide or protein expressed in a cancer cell.

For example, disclosed are compositions, wherein the antigen comprises a cancer cell antigen. The antigen can be an antigen that includes a peptide or protein or other molecule, such as nucleic acid or sugar, derived from a cancer. Derived from means that the molecule is a molecule found as part of the cancer cell or typically considered as part of the cancer cell. Disclosed are cancer cell antigens derived from a Hodgkins lymphoma cell, non-Hodgkins lymphoma cell, B cell lymphoma cell, T cell lymphoma cell, myeloid leukemia cell, leukemia cell, mycosis fungoides cell, carcinoma cell, squamous cell carcinoma cell, adenocarcinoma cell, sarcoma cell, glioma cell, blastoma cell, neuroblastoma cell, plasmacytoma cell, histiocytoma cell, melanoma cell, adenoma cell, hypoxic tumour cell, myeloma cell, AIDS-related lymphoma cell, AIDS related sarcoma cell, metastatic cancer cell, bladder cancer cell, brain cancer cell, nervous system cancer cell, squamous cell carcinoma cell, neuroblastoma cell, glioblastoma cell, ovarian cancer cell, skin cancer cell, liver cancer cell, melanoma cell, colon cancer cell, cervical cancer cell, cervical carcinoma cell, breast cancer cell, epithelial cancer cell, renal cancer cell, genitourinary cancer cell, pulmonary cancer cell, esophageal carcinoma cell, hematopoietic cancer cell, testicular cancer cell, colo-rectal cancer cell, prostate cancer cell, or pancreatic cancer cell.

5. Common Attributes of the Compositions

a) Sequence Similarities

It is understood that one way to define any known variants and derivatives or those that might arise, of the disclosed genes and proteins herein is through defining the variants and derivatives in terms of homology to specific known sequences. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. For example, the homology can be calculated after aligning the two sequences so that the homology is at its highest level.

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

b) Peptides

(1) Protein Variants

As discussed herein there are numerous variants of the disclosed antibodies that can be produced given the specific antibodies disclosed herein. As antibodies are proteins, techniques for protein modification and variant production are applicable to the disclosed antibodies as well as other peptides or proteins and these techniques are known and herein contemplated. In addition to the disclosed functional hK2 antibodies, there are derivatives of the hK2 antibodies which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations | |
|---|---|---|
| alanine | Ala | A |
| allosoleucine | AIle | |
| arginine | Arg | R |
| asparagine | Asn | N |
| aspartic acid | Asp | D |
| cysteine | Cys | C |
| glutamic acid | Glu | E |
| glutamine | Gln | K |
| glycine | Gly | G |
| histidine | His | H |
| isolelucine | Ile | I |
| leucine | Leu | L |
| lysine | Lys | K |
| phenylalanine | Phe | F |
| proline | Pro | P |
| pyroglutamic acid | pGlu | |
| serine | Ser | S |
| threonine | Thr | T |
| tyrosine | Tyr | Y |
| tryptophan | Trp | W |
| valine | Val | V |

TABLE 2
Amino Acid Substitutions

| | Exemplary Conservative |
|---|---|
| | Substitutions, others are |
| Original Residue | known in the art. |
| Ala | ser |
| Arg | lys, gln |
| Asn | gln; his |
| Asp | glu |
| Cys | ser |
| Gln | asn, lys |
| Glu | asp |
| Gly | pro |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the disclosed monoclonal antibodies. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least a given % homology, for example, 70% homology, to a particular sequence wherein the variants are conservative mutations.

As this specification discusses various proteins and protein sequences, such as antibodies, particularly monoclonal antibodies, it is understood that the nucleic acids that can encode them are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is also understood that while no amino acid sequence indicates what particular DNA sequence encodes that protein within an organism, where particular variants of a disclosed protein are disclosed herein, the known nucleic acid sequence that encodes that protein in the particular cell from which that protein arises is also known and herein disclosed and described.

c) Delivery of the Compositions to Cells

The disclosed compositions can be delivered to cells or organisms typically using any method for performing these functions. For example, the antibodies themselves may be delivered to an organism using for example, a direct injection of the antibody or other means, or the antibody may be delivered to the organism via a cell for example that expresses the antibody. For example, the nucleic acids which encode the disclosed antibodies may be used to produce cells which express the antibody. The nucleic acids may delivered as disclosed herein or by comparable method.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991)Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

(1) Nucleic Acid Based Delivers Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as nucleic acids encoding hK2 or an antibody to hK2, into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(a) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol , and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the. insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(b) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to fonn new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291(1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wiclcham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(c) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

The vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(d) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8:33-41, 1994; Cotter and Robertson,.Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

(2) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed compositions or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconiugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other speciifc cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of inurine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

(3) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

d) Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(1) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273:113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78:993 (1981)) or 3′ (Lusly, M. L., et al., Mol. Cell Bio. 3: 1108 (1983)) to the transcription unit. Furthermore, enhancers can be within an intron (Banerji, J. L. et al., Cell 33:729 (1983)) as well as within the coding sequence itself (Osborne, T. F., et al., Mol. Cell Bio. 4:1293 (1984)). They are usually between 10 and 300 bp in length, and they function in cis. Enhancers fimction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or allcylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eulcaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the MRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like MRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

(2) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1:327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

e) Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transderrnally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be detennined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconiugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffier, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biochimica Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of finctions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

(1) Pharmaceutically Acceptable Carriers

The compositions, including antibodies, can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

(2) Therapeutic Uses

The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of sildl in the art. The dosage can be adjusted by the individual physician in the event of any counterindications. Dosage can vary, and can be administered in one or more dose administrations daily, for one or several days.

Other hK2 binding compositions, such as antibodies, which do not have a specific pharmacuetical function, but which may be used for tracking changes within cellular chromosomes or for the delivery of diagnositc tools for example can be delivered in ways similar to those described for the pharmaceutical products.

The hK2 binding compositions can also be used for example as tools to isolate and test new drug candidates for a variety of diseases.

f) Chips and Micro Arrays

Disclosed are chips where at least one address is a disclosed composition herein, such as a monoclonal antibody that preferentially binds hK2, or fragments thereof. Also disclosed are chips where at least one address is the sequences or portion of sequences set forth in any of the peptide molecules or nucleotide molecules disclosed herein.

Also disclosed are chips where at least one address is a variant of the compositions or part of the compositions set forth in herein.

6. Computer Readable Mediums

It is understood that the disclosed nucleic acids and proteins, for example monoclonal antibodies that preferentially bind hK2, can be represented as a sequence consisting of the nucleotides or amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

7. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for assessing a subject's risk for acquiring prostate cancer, comprising any of the disclosed antibodies is disclosed.

8. Compositions with Similar Functions

It is understood that the compositions disclosed herein have certain functions, such as specifically binding hK2 or inhibiting or enhancing the function of hK2. Disclosed herein are certain structural requirements for performing the disclosed functions, and it is understood that there are a variety of structures which can perform the same function which are related to the disclosed structures, and that these structures will ultimately achieve the same result, for example stimulation or inhibition of hK2.

C. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1 Plus DNA, synthesizer (for example, Model 8700 automated synthesizer of Milligen-Biosearch, Burlington, MA or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert -butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W. H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide--thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J.Biol.Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton RC et al., Techniques in Protein Chemistry IV. Academic Press, New York, pp. 257-267(1992)).

3. Processes for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence encoding the disclosed monoclonal antibodies and a sequence controlling the expression of the nucleic acid.

Also disclosed are nucleic acid molecules produced by the process comprising linking in an operative way a nucleic acid molecule comprising a sequence having 80% identity to a composition set forth herein, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence encoding, for example, the disclosed monoclonal antibodies, and a sequence controlling the expression of the nucleic acid.

Disclosed are nucleic acid molecules produced by the process comprising linking in an operative way a nucleic acid molecule comprising a sequence encoding a disclosed peptide and a sequence controlling an expression of the nucleic acid molecule.

Disclosed are nucleic acid molecules produced by the process comprising linking in an operative way a nucleic acid molecule comprising a sequence encoding a peptide having 80% identity to a peptide such as the disclosed monoclonal antibodies, and a sequence controlling an expression of the nucleic acid molecule.

Disclosed are nucleic acids produced by the process comprising linking in an operative way a nucleic acid molecule comprising a sequence encoding a peptide having 80% identity to a disclosed herein, wherein any change from the peptides disclosed herein are conservative changes and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

D. Methods of Using the Compositions

1. Methods of Using the Compositions as Research Tools

The disclosed compositions including, for example, monoclonal antibody 6B7, can be used in immunohistochemistry, and its staining pattern appeared similar to 8A3 and G586, two of the few previously reported monoclonal antibodies reactive with hK2 [Darson et al., Urology 1997; Tremblay et al., Am J Pathol 1997]. The antibodies 6B7 and 11C4 recognize two discrete epitopes and can be used in a sandwich ELISA assay to detect secreted hK2 antigen.

The disclosed compositions can be used in a variety of ways as research tools. For example, the disclosed compositions, such as the monoclonal antibodies can be used to study the interactions involving hK2, by for example acting as inhibitors of binding.

The compositions can be used for example as targets in combinatorial chemistry protocols or other screening protocols to isolate molecules that possess desired functional properties related to the disclosed monoclonal properties.

The disclosed compositions can also be used diagnostic tools related to diseases such as cancer, such as prostate cancer and breast cancer. For example, methods of diagnosing a susceptibility to cancer comprising, reacting a subject's tissue, such as blood, with the any of the disclosed compositions and determining the level of hK2 present in the subject's tissue, such as blood, are disclosed.

The disclosed compositions can be used as an adjuvant to generate an immune response to an antigen. Also disclosed are methods of enhancing an immune response to an antigen comprising administering to a subject a tumor cell line wherein the tumor cell line comprises a vector that encodes an antigen.

Also disclosed are methods further comprising modulating the immune response to an antigen by depleting IL-4, meaning a reduction in amount or numbers of a molecule or composition. Depleting can mean any amount or number less than the original including but not limited to the complete absence of the molecule or composition. Depleting can also occur because the number of enzymatically active molecules have been reduced. In this situation the molecule is present but no longer active.

Also disclosed are methods of diagnosing a susceptibility to cancer comprising reacting a subject's tissue with any of the disclosed compositions and determining the level of hK2 present in the subject's tissue. An increase in hK2 indicates an increased susceptibility for acquiring cancer or having a negative prognosis for reducing the cancer, if already acquired. The disclosed compositions and methods can be used in combination with any other assay related to cancer, such as assays to diagnose cancer based on PSA levels.

Also disclosed are methods, further comprising comparing the level of hK2 in the subject relative to a standard. The standard can be derived from a norm created across a specific population or set of individuals. It is understood that a variety of tissues can be assayed for hK2, such as blood, colon, prostate, gastrointestinal, muscle, bone, and lymph, for example.

The amount of hK2 present can be assessed using any method. Any method that employs the disclosed compositions or employs the understanding imparted by the disclosed compositions can be used. For example, ELISA assays, ELIspot assays, radioimmunoassays, Imnunocytochemistry assays, or chemiluminescence assays can be used and developed.

Disclosed are assays that utilize the binding properties of the disclosed compositions to preferentially recognize hK2 rather than PSA.

Disclosed are methods of modulating the enzymatic activity of hK2. Also disclosed are methods of inhibiting the enzymatic activity of hK2, comprising contacting hK2 with a composition that inhibits hK2. Also disclosed are methods of enhancing the enzymatic activity of hK2, comprising contacting hK2 with a composition that enhances hK2.

Disclosed are methods of modulating the enzymatic activity of PSA. Also disclosed are methods of inhibiting the enzymatic activity of PSA comprising contacting hK2 with and inhibitor of hK2. Also disclosed are methods of enhancing the enzymatic activity of PSA comprising contacting hK2 with a compositions that enhances hK2. The disclosed compositions can be particularly useful in detection of different forms of antigen, since the disclosed compositions, such as certain monoclonal antibodies inhibit or enhances the enzymatic activity.

In certain embodiments the diagnostic methods are performed on subject's that may be susceptible to cancer, or already have some level of cancer. Also disclosed are methods, wherein the cancer is either prostate cancer or breast cancer. It is also understood that methods are disclosed which are designed to detect the staging of a particular cancer, such as prostate cancer or breast cancer.

The disclosed compositions can be used as discussed herein as either reagents in micro arrays or as reagents to probe or analyze existing microarrays or make new micro arrays. The compositions can also be used in any known way of using the computer readable embodiments of the disclosed compositions, for example, to study relatedness or to perform molecular modeling analysis related to the disclosed compositions.

2. Pharmaceutical Methods

A method of reducing the proliferation of cancer cells comprising administering any of the disclosed compositions to the cancer cells. Disclosed are methods wherein, the compositions alter the function of hK2, by for example, inhibiting or enhancing the hK2 function.

Also disclosed are methods, wherein the cancer cells are contained within a patient.

Thus, in certain embodiments, the disclosed compositions can be used for the treatment or the prevention of cancer. Disclosed are methods, wherein the cancers are prostate or breast cancer.

3. Methods of Producing Antibodies

Disclosed are methods of using cells, such as tumor cells, to present antigens in vivo or to be used in combination with antigens. This method can be used to generate antibodies, such as monoclonal antibodies. For example, using cDNA expressed in tumors (tumor cell lines), hybridomas producing monoclonal antibodies specific for the cDNA's product have been efficiently made. The monoclonal antibodies can be modified or used in any of the ways described herein or known in the art. The cDNA expressed can encode hK2, and the monoclonal antibody produced can be hK2-specific. Any other antigen for which an antibody is desired can be used in this method. Examples of tumors and tumor cell lines that can be used to express the antigen for the generation of antibodies are numerous. They include, but are not limited to L1, EMT6, or CT-26 cells. For example, as shown in Example 2, the tumor is continually producing the native antigen in vivo. The increasing amount and continuous release of antigen as well as the growing tumor itself acts as an adjuvant to increase the antibody response. Thus, the tumor can act both as the delivery vehicle and the adjuvant.

The ability of tumors to act as adjuvants can also have direct practical applications. First, advances in molecular biology and genomics have led to the identification of large numbers of genes whose products are poorly characterized. The antibodies made by this method are extremely useful for the characterization of the expression of the protein in different cell types and in determining subcellular expression levels, and, equally important, they can also serve as tools to elucidate the function of these molecules. Second, elucidation of the characteristics that make certain tumors strong adjuvants could provide important information for generating effective anti-tumor immunity.

E. Sequences

1. SEQ ID NO:3, hK2

ORIGIN

| | |
|---|---|
| 1 | ATGTGGGACC TGGTTCTCTC CATCGCCTTG TCTGTGGGGT GCACTGGTGC CGTGCCCCTC |
| 61 | ATCCAGTCTC GGATTGTGGG AGGCTGGGAG TGTGAGAAGC ATTCCCAACC CTGGCAGGTG |
| 121 | GCTGTGTACA GTCATGGATG GGCACACTGT GGGGGTGTCC TGGTGCACCC CCAGTGGGTG |
| 181 | CTCACAGCTG CCCATTGCCT AAAGAAGAAT AGCCAGGTCT GGCTGGGTCG GCACAACCTG |
| 241 | TTTGAGCCTG AAGACACAGG CCAGAGGGTC CCTGTCAGCC ACAGCTTCCC ACACCCGCTC |
| 301 | TACAATATGA GCCTTCTGAA GCATCAAAGC CTTAGACCAG ATGAAGACTC CAGCCATGAC |
| 361 | CTCATGCTGC TCCGCCTGTC AGAGCCTGCC AAGATCACAG ATGTTGTGAA GGTCCTGGGC |
| 421 | CTGCCCACCC AGGAGCCAGC ACTGGGGACC ACCTGCTACG CCTCAGGCTG GGGCAGCATC |
| 481 | GAACCAGAGG AGTTCTTGCG CCCCAGGAGT CTTCAGTGTG TGAGCCTCCA TCTCCTGTCC |
| 541 | AATGACATGT GTGCTAGAGC TTACTCTGAG AAGGTGACAG AGTTCATGTT GTGTGCTGGG |
| 601 | CTCTGGACAG GTGGTAAAGA CACTTGTGGG GGTGATTCTG GGGGTCCACT TGTCTGTAAT |
| 661 | GGGGTGCTTC AAGGTATCAC ATCATGGGGC CCTGAGCCAT GTGCCCTGCC TGAAAAGCCT |
| 721 | GCTGTGTACA CCAAGGTGGT GCATTACCGG AAGTGGATCA AGGACACCAT CGCAGCCAAC |
| 781 | CCCTGA |

### F. EXAMPLES

Unless indicated otherwise, parts are parts by weight, temperature is in ° C. or is at ambient temperature, and pressure is at or near atmospheric.

### 1. Example 1 hK2 Antibodies

a) Materials and Methods

(1) Construction of hK2-Expression Vector:

The mRNA for hK2 was isolated from the human prostate cancer cell line, LNCaP (ATCC, Manassas, Va.) using TRIZOL Reagent (GIBCO, Grand Island, N.Y.). An hK2 cDNA was isolated using reverse transcriptase/polymerase chain reaction (RT-PCR) using the following hK2-specific primers:

(SEQ ID NO:1)

5′ Primer (5′ CATAGGTCGACCTGTGTCAGCATGTGGGA 3′)

(SEQ ID NO:2)

3′ Primer (3′ CATAGAAGCTTCACTCAGGGGTTGGCTGC 5′)

The PCR product was subcloned into the pBluescript II vector (Stratagene, La Jolla, Calif.) using Sal I and Hind III sites designed into the primers, and clones were isolated and analyzed by restriction mapping and DNA sequencing. The hK2 cDNA was subcloned into pHβ-apr-1-neo (Gunning P, et al., Proc Natl Acad Sci U S A 1987;84:4831-5) to make the hK2 mammalian expression plasmid, pHβ-hK2-neo.

(2) Cell Lines and hK2-Transfectants:

pHβ-hK2-neo was used to transfect line 1 (L1), a BALB/c (H-2^{d}) small cell lung carcinoma (Yuhas J M, et al., Cancer Res 1974;34:722-8), and P815, a DBA/2 (H-b 2^{d}) mastocytoma cell line (Lundak RL, Raidt D J., Cell Immunol 1973;9:60-6) as previously described (Wei C, et al., Cancer Immunol Immunother 1996;42:362-), in order to generate L1/hK2 and P815/hK2, respectively. Briefly, 1×10⁵ tumor cells were grown overnight and transfected the next day with 2.5 ug DNA using LIPOFECTIN (GIBCO) and selected using Geneticin (G418) (GIBCO) at 400 ug/ml. Resistant cells were cloned by limiting dilution and screened via RT-PCR for mRNA. RT-PCR was performed as above except RNA from transfected cell lines was used as the original template. Mouse β-actin primers were used as controls for the integrity of the RNA and reverse-transcriptase (RT) was omitted from certain reactions to confirm that the product was amplified from mRNA and not contaminating DNA. To generate the L1/hK2/B7 cell line that was used as the immunization for the fusion, L1/hK2 was transfected with pHβ-B7-1-gpt (Yeh K Y, et al., Cell Immunol 1995;165:217-24), which is an expression vector which drives the expression of the mouse B7-1 costimulatory molecule under the gpt selectable marker. This cell line was shown to be positive for mouse B7-1 by flow cytometry using a FITC-conjugated anti-mouse B7-1 monoclonal antibody (PharMingen, San Diego, Calif.). The L1/PSA/IL-2 cell line was described previously (Turner M J, et al., J Immunol Methods 2001;256:107-19). All cell lines were grown in MAT/P cell culture media containing 2% fetal calf serum (Hyclone, Logan, Utah).

(3) Immunization of Mice to Generate Monoclonal Antibodies

Mice used were either male BALB/cByJ (Jackson Laboratory, Bar Harbor, Me.), male PSA transgenic mice (BALB/c.PSA) (Wei C, et al., Proc Natl Acad Sci USA 1997;94:6369-74), or male non-transgenic littermates. In analyzing the antibody response from serum, mice were injected intraperitoneally (i.p.) with 1×10⁷ irradiated (30 Gy) L1/hK2 cells at 2 week intervals. Serum was taken 9-13 days after each immunization and tested for reactivity to both hK2 and PSA. To generate monoclonal antibodies, a BALB/c.PSA transgenic mouse was injected once intramuscularly with 8×10⁵ live L1/hK2/B7 cells and the spleen was removed 20 days later for the B cell fusion.

(4) B Cell Fusion

The fusion was performed basically as described previously (Harwell L W, et al., J Immunol Methods 1984;66:59-67). In brief, the spleen was fused with P3 myeloma cells (Kearney J F, et al., J Immunol 1979;123:1548-50) at a 1:1 ratio using 35% polyethyleneglycol (Sigma, St. Louis, Mo.). The fused cells were cultured for 2 days at which time ⅔ of the fusion was cryopreserved and the remainder was plated in eight 96-well plates. Growth-positive wells were screened 11-15 days later by ELISA for hK2-reactive antibodies (see below). Wells which showed reactivity approximately 10 fold or greater above background were then subcloned and rescreened against PSA.

(5) Antibody ELISA

An antibody ELISA was developed that can detect either anti-PSA or anti-hK2 antibodies from the same serum or hybridoma supernatant (SN). The plate was coated with 3 μg/ml of rabbit anti-human PSA (DAKO, Carpinteria, Calif.) for 1 hour at 37° C. and then blocked overnight at 4° C. in blotto-tween (PBS containing 5% powdered non-fat milk and 0.2% Tween 20). The antigen, either native PSA (Calbiochem, San Diego, Calif.), SN from P815/PSA, or SN from P815/hK2 tumor cell lines, was then added and incubated for 1 hour at 37° C. The test sample was then added, either serum from tumor-immunized mice or hybridoma SN, for 2 hours at 37° C. A rabbit-anti-mouse IgG-horseradish peroxidase (HRP) (Jackson Immunoresearch, West Grove, Pa.) was then added for 1 hour at 37° C., and developed using O-phenylenediamine (OPD) (Sigma) as the chromogen for 10-15 minutes. The absorbance at 490 nm was determined using a Dynascan MRX ELISA reader.

(6) hK2-Specific Sandwich ELISA

Purified 6B7 (anti-hK2 monoclonal antibody, clone 6B7) was coated onto an ELISA plate at 3 ug/ml for 1 hour at 37° C. Samples were added next for 2 hours at 37° C. and included SN from transfected cell lines, purified PSA from Cortex (San Leandro, Calif.) or Calbiochem, and transgenic mouse prostate solubilized in 1% NP40 lysis buffer using a pellet pestle (Kontes, Vineland, N.J.). A second anti-hK2 monoclonal antibody, clone 11C4, was added at a concentration of 3 μg/ml, detected using a goat anti-mouse IgG3-HRP (Southern Biotech, Birmingham, Ala.), and developed similarly to the antibody ELISA using OPD.

(7) ELIspot Assay

A MAIP ELIspot 96-well plate (Millipore, Bedford, Mass.) was coated with H117, a monoclonal antibody which is reactive with PSA and hK2 (Abbott Labs, Abbott Park, Ill.) (Lovgren J, et al., Biochem Biophys Res Commun 1995;213:888-95), at 3 μg/ml at 37° C. The plate was blocked overnight at 4° C. in blocking buffer (PBS containing 2% FBS, 5 mM HEPES, and 0.05% sodium azide), followed by the addition of tumor cells for 8 hours at 37° C. The secondary antibody was either 1F8-biotin (hK2-specific) or H50-biotin (hK2/PSA reactive) (Abbott Labs) (Lovgren J, et al., Biochem Biophys Res Commun 1995;213:888-95)and was added overnight at 4° C., followed by the detecting antibody, streptavidin-alkaline phosphatase (Southern Biotech), and development of the assay with 5-bromo-4-chloro-3-indolyl phosphate/nitro blue tetrazolium (BCIP/NBT) reagent (Sigma).

(8) Human Prostate Staining

Immunohistochemical stains were performed on formalin-fixed, 4-5 micron thick, paraffin-embedded tissue sections using standard protocols (Bourne J. Handbook of Immunoperoxidase Staining Methods. Santa Barbara: Dako Corp., 1983), with the following specifications. Antigen unmasking with heat retrieval was accomplished in citrate buffer pH 6 by placing slides in a microwave (1500 watts at power level 6) pressure cooker for 30 minutes followed by a 15 minute cool down. Sections were stained for 45 minutes using 6B7 at a final concentration of 18 μg/ml, followed by 20 minute incubations in horse anti-mouse IgG-biotin (Vector Laboratories, Inc. Burlingame, Calif.) and Streptavidin-HRP (Jackson Lab). Slides were developed with aminoethylcarbazole (AEC+) (Dako) for 7 minutes and counterstained in Mayers Hematoxylin.

(9) Purification of hK2 and Enzymatic Assay

Supernatant from P815-hK2 was purified over a 6B7 affinity column using standard procedures (Wofsy L, Burr B., J Inununol 1969;103:380-2). The fractions containing hK2 were pooled and IGEPAL (Sigma) was added to a final concentration of 0.1%. The pooled fractions were concentrated in Centricon-10 spin columns (Millipore, Bedford, Mass.). The enzymatic activity of hK2 was determined using the chromogenic substrate H-D-prolyl-L-phenylalanyl-L-arginine-p-nitroaniline-dihydrochloride (S-2302, Chromogenix, Diapharma Group Inc, Westchester, Ohio) (Mikolajczyk SD et al., Eur J Biochem 1997;246:440-6). Purified hK2 was incubated with 1 mM S-2302 in 0.1 M Tris-HCl, pH 7.8, 0.1 M NaCl in a final volume of 150 ul that contained 0.1% bovine serum albumin. The reactions were performed in U-bottom microtiter plates at 37° C. The release of p-nitroanilide was followed at 405 nm using a Dynascan MRX ELISA reader. When antibodies were included in the assay, the purified antibody and hK2 were incubated for 30 minutes at 37° C. prior to the addition of S-2302. Purified antibodies were buffer exchanged in PBS before use.

b) Results

(1) Generation and Characterization of hK2-Expressing Cell Lines

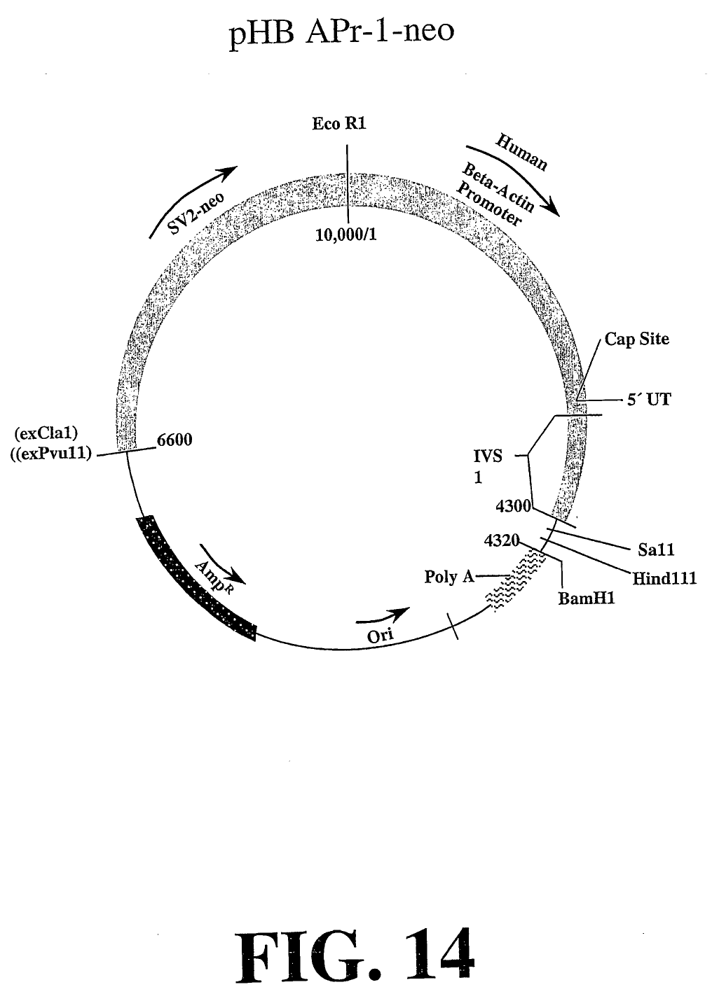

In order to create hK2 expressing tumor cell lines, a hK2 cDNA was generated by reverse transcriptase-mediated PCR using hK2-specific primers and MRNA isolated from the human prostate cancer cell line LNCaP. This cDNA was cloned into pBluescript and the authenticity of the clone was confirmed by DNA sequencing, matching the published sequence of hK2 (Young C Y, et al., Biochemistry 1992;31:818-24). The sequence is set forth in SEQ ID NO:3. The vector is shown in FIG. 14.

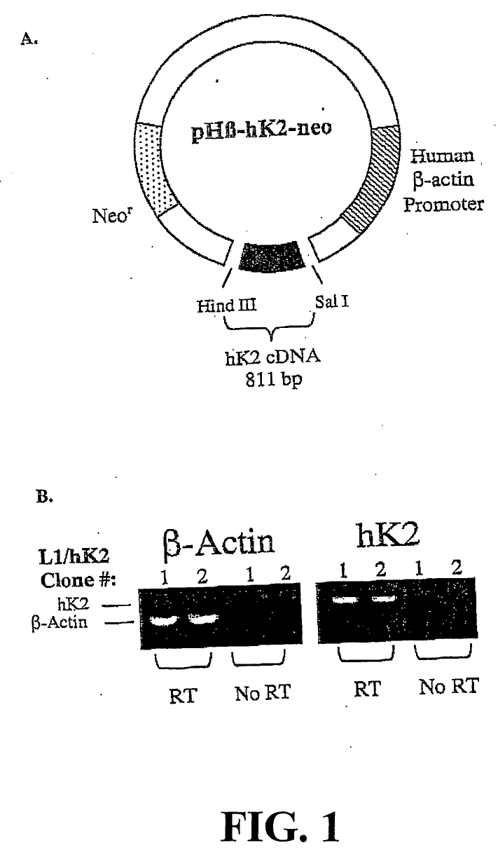

The verified hK2 cDNA was subcloned into the human β-actin expression vector (FIG. 1A), transfected into Line 1 (L1) tumor cells using lipofectin and transfectants selected using G418. The resulting G418 resistant clones were screened for mRNA encoding hK2 by RT-PCR analysis using hK2-specific primers to determine whether hK2 mRNA was produced by the transfected cells. A DNA product of the predicted size of hK2 (811 base pairs) was detected in two independent clones (FIG. 1B). β-actin primers were used as controls to illustrate the integrity of the mRNA and as a positive control for the RT-PCR reactions. No product was seen if the reverse transcriptase was omitted from the RT-PCR procedure indicating that the product was amplified from the mRNA.

(2) Immunization of Mice and Analyses of Serum Antibody Response to hK2

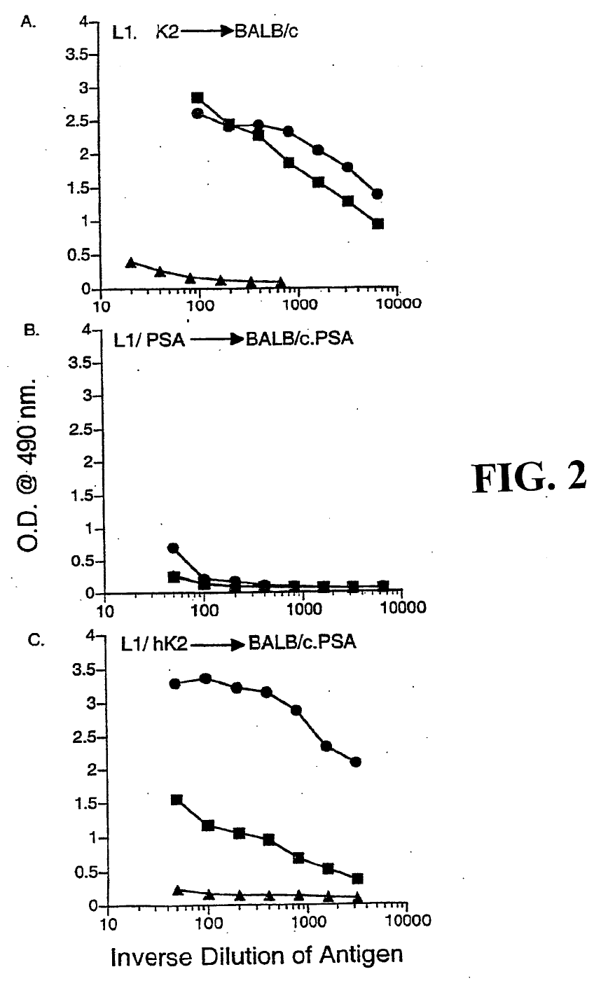

BALB/c and BALB/c.PSA transgenic mice were immunized at 2-week intervals four times intraperitoneally (i.p.) with irradiated hK2-transfected tumor cells and screened by ELISA 10-14 days after the final immunization. For these analyses, a rabbit anti-PSA polyclonal antibody was used as the capture antibody in the ELISA with the rationale that it would react with both hK2 and PSA because of their extensive homology. By varying the antigen (e.g. using either PSA or hK2), the assay could be used to detect mouse antibodies reactive with either PSA or hK2, which in turn would be detected with a horseradish peroxidase (HRP)-coupled rabbit anti-mouse immunoglobulin. Sera from mice that had been immunized with tumors expressing either PSA, hK2, or with the parental Line 1 tumor cell line, were tested for antibody responses to PSA or hK2. FIG. 2A demonstrates that L1/hK2 tumors elicit a high titer antibody response in BALB/c mice. The serum reactivity shows substantial reactivity to both PSA and hK2. Similar results were obtained following earlier immunizations, with live tumor immunization, or using non-transgenic littermates instead of BALB/c mice. Finding substantial reactivity towards both hK2 and PSA in BALB/c mice is consistent with the high degree of sequence identity (80% identical) between PSA and hK2. Interestingly, the BALB/c.PSA transgenic mice immunized with tumor expressing PSA (L1/PSA) did not make an antibody response toward PSA, as shown in FIG. 2B, presumably due to immunological tolerance. L1/PSA produces approximately the same amount of antigen as does L1/hK2 (20-50 ng/ml) and generates a substantial antibody response to PSA and hK2 in BALB/c mice. Perhaps most striking, when the PSA transgenic mice were immunized with L1/hK2, they generated a very high titered response towards hK2, but showed much less reactivity towards PSA (FIG. 2C). These results indicate that mice immunized with tumors expressing hK2 can be a source of hK2-specific reagents.

(3) Generation of hK2-Reactive Monoclonal Antibodies

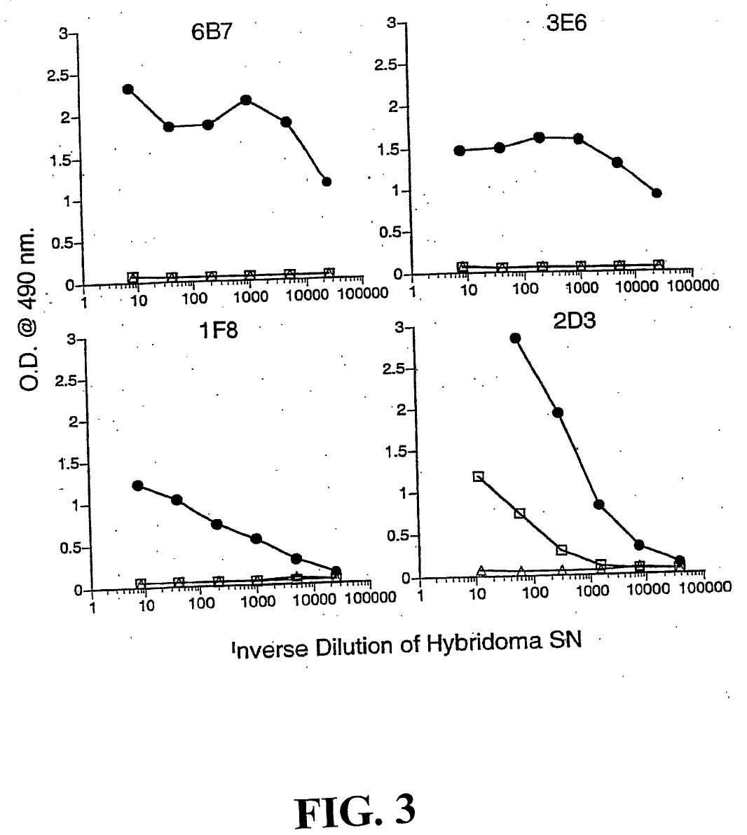

A fusion was performed from the spleen of a BALB/c.PSA transgenic mouse that was immunized with live hK2-expressing tumor. The fusion was initially screened for reactivity toward hK2, positive clones subcloned, and subsequently analyzed for reactivity towards PSA. Representative ELISA results from these analyses are shown in FIG. 3 and illustrate that the hK2-reactive antibodies 6B7, 3E6, and 1F8 did not appear to cross-react with PSA. Similar results were obtained with 27 additional monoclonal antibodies. Interestingly, consistent with the serum data in FIG. 2, it was found that some hybridomas (e.g. 2D3) did react with both hK2 and PSA. Nevertheless, the fusion resulted in a predominance of hK2-specific, non-PSA cross-reactive hybridomas (30/42 or 71%). From the initial screen of 30 hK2-reactive hybridomas, 11 have been isotyped and characterized further. Of this panel of 11, most (8) were IgG1, but other isotypes were also obtained, including 2 IgG3s (11C4 and 9B4) and 1 IgG2b (1F8).

(4) Demonstration of Specificity: hK2-Specific Sandwich ELISA and ELIspot

ELISA and ELIspot assays were developed to detect hK2 and determine the reactivity towards PSA. A sandwich ELISA using two of the hK2-reactive monoclonal antibodies was developed utilizing 6B7 as the capture antibody and 11C4 as the detecting antibody. The assay was evaluated with hK2 and several sources of PSA to examine its specificity (FIG. 4A). P815/hK2 SN at a concentration of approximately 200 ng/ml is positive and is detectable to approximately 3 ng/ml. Several different sources of PSA were used in this assay and none showed reactivity. For example, purified PSA obtained from Calbiochem or Cortex at 50 ng/ml and recombinant PSA from tumor cell line SN (L1/PSA/IL-2) at 700 ng/ml was negative. In order to determine if the antibodies would react with PSA at even higher concentrations, a sample of homogenized prostate from a transgenic mouse that contained PSA at a concentration of 300 ug/ml was tested and was completely negative by this assay (dotted line in FIG. 4), illustrating that this assay does not react with PSA even at extremely high amounts of antigen. Together with the estimate that the 6B7/11C4 pair can detect hK2 at a concentration of approximately 3ng/ml, the PSA cross-reactivity of this assay is less than 3/300,000 or 0.001%.

An ELIspot analysis using one of the hK2-reactive antibodies, 1F8, was performed to examine further the specificity of the antibody for PSA or hK2 (FIG. 4B). Tumor cells that secrete antigen (either PSA or hK2) were grown in an ELIspot plate, allowing them time to secrete a high local concentration of antigen. This assay detects antigen, either PSA or hK2, secreted by tumor cells, and the “spots” represent tumor cells that have secreted their antigen at a precise location on the filter of the ELIspot plate. This high local concentration of antigen is ideal for assessing the specificity of the reaction towards hK2 and PSA. In these experiments the H117 antibody, which is known to react with a shared determinant on PSA and hK2 (Lovgren J, et al., Biochem Biophys Res Commun 1995;213:888-95), was used as a capture antibody. L1/PSA, L1/hK2 or L1 cells were plated at various densities. Antigen secreted by the cells was captured by the H117 antibody and the antigen was detected with either biotinylated-1F8 (hK2-reactive) or biotinylated-H50 (reactive with either PSA or with hK2). Neither the H117/1F8 nor the H117/H50 combination showed reactivity in the wells containing only the parental L1 cells. Both the L1/PSA and L1/hK2 cells were detected using the H117/H50 pair. In striking contrast, the H117/1F8 pair only detected the L1 cells transfected to express hK2. No reactivity was seen from the wells that contained the PSA secreting cells using the H117/1F8 pair. Since the H117 clearly binds the PSA, all the specificity in this assay is due to the 1F8 monoclonal antibody. Thus, in this assay there is no detectable reactivity of 1F8 with PSA. This assay has also been performed using 3H2 and 11C4 as the detecting antibody, with similar results.

(5) Use of 6B7 in Immunohistochemistry

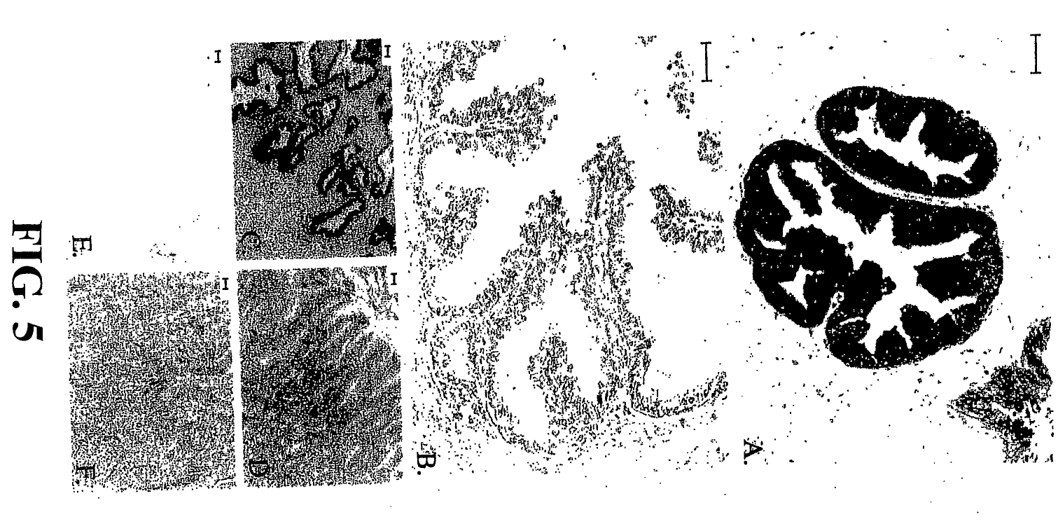

The antibodies disclosed herein work in immunohistochemistry assays. 6B7 appeared to react with human prostate paraffin embedded sections, and a more extensive analysis was performed. As can be seen in FIG. 5A & C, the monoclonal antibody clearly stains the epithelial cells of the prostate, shown by the reddish precipitate on the luminal sides of the glandular structures. For comparison, an IgG1 isotype control done at the same time is illustrated in FIG. 5B. Interestingly, some heterogeneity in staining of these cells was observed. This antibody showed no staining in several other tissues (FIG. 5D-F) including stomach, muscle, and kidney, which supports the primarily prostate-specific expression of hK2.

(6) Functional Significance of hK2-Specific Monoclonal Antibodies

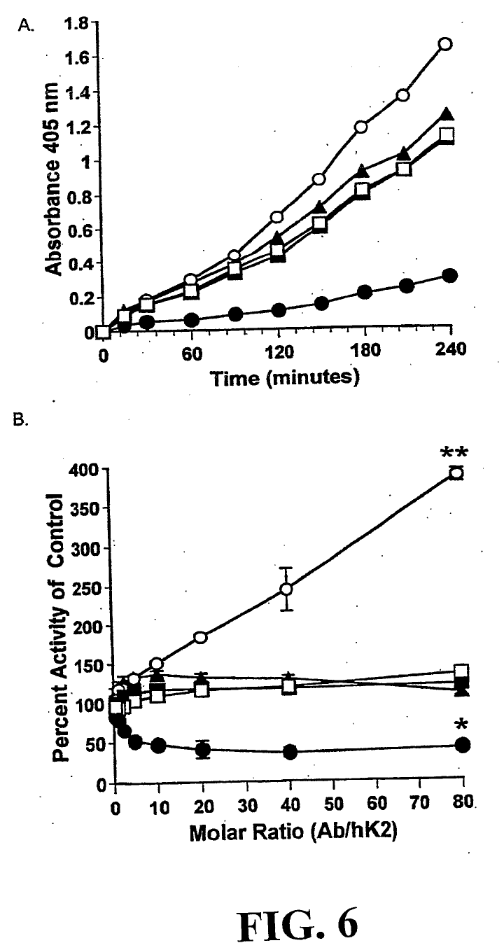

Mikolajczyk et al. (Mikolajczyk S D, Int J Cancer 1999;81:438-42) has shown previously that the chromogenic substrate S-2302 can be used to analyze the specific enzymatic activity of hK2. An in vitro hK2 enzymatic assay was performed as described herein, and an initial survey was conducted with 11 hK2-specific antibodies to determine their effect on hK2 activity. In this survey, 9 of the antibodies showed little effect whereas one of the monoclonals (1F8) showed enhancement of activity, and another (3C7) showed inhibition of enzymatic activity. These two antibodies, as well as one that showed little effect on activity (6B7), were investigated further. In FIG. 6A, purified antibodies were incubated with 50 ng of hK2 at a 20-fold molar excess, and the absorbance increase at 405 nm was followed over time. These data illustrate that one of the antibodies, 1F8, enhanced the activity of hK2 to a level approximately 50% greater than the IgG2b isotype control. In contrast, another monoclonal, 3C7, showed substantial blocking of the enzymatic activity of hK2, with an 80% reduction as compared to the IgG1 isotype control. As in the initial survey, 6B7 has little effect on the biological activity of hK2, as the hydrolysis of substrate with this antibody is similar to the incubation with the corresponding isotype control antibodies. In order to test the dependence on the concentration of antibody, a similar assay was performed varying the antibody concentration while keeping the amount of hK2 constant (25 ng), and results are shown in FIG. 6B. These data are expressed as percent activity of hK2 when incubated without antibody and is plotted versus the molar ratio of antibody to hK2. At 8 hours, 1F8 seems to enhance the activity of hK2 380% at a 80:1 molar ratio of antibody to enzyme. At an 80:1 molar ratio of antibody to enzyme, 3C7 can inhibit the activity of hK2 to 35% of control and has inhibited to as low as 15% in other assays (data not shown). At a 5:1 molar ratio of antibody to enzyme, 3C7 can inhibit the activity of hK2 to 50% of control. Again, incubation of hK2 with any concentration of 6B7 has little effect on the enzymatic activity of hK2. These results demonstrate that although most of the hK2-specific monoclonal antibodies developed do not alter the activity of hK2, one antibody that enhances and one antibody that inhibits the enzymatic activity of hK2 were identified.

(7) Additional Characterization of 3C7 Inhibition

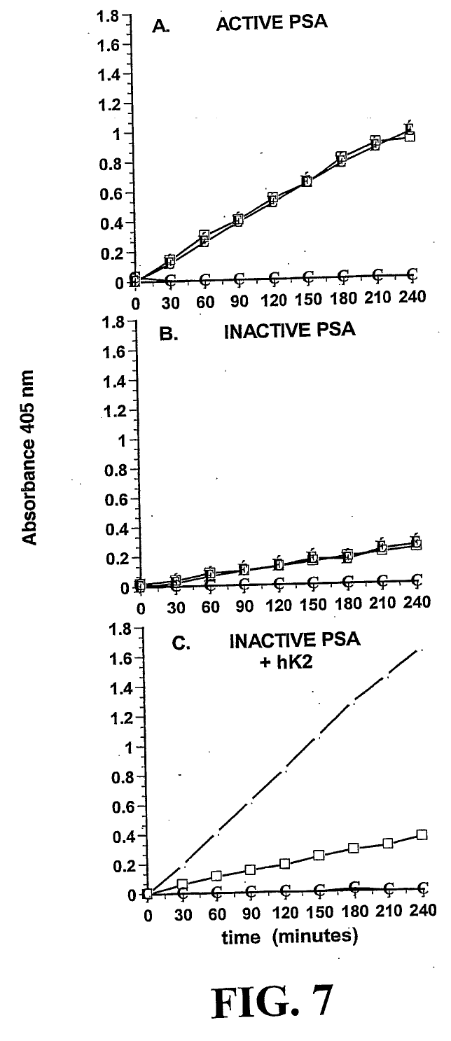

3C7 could inhibit the activity of hK2 on its chromogenic substrate. In the current line of investigation, it was investigated whether the activity of hK2 on a protein substrate could be blocked. In particular, it was shown that blocking hK2 could inhibit its activation of other enzymes. In the current study, whether 3C7 could inhibit the activity of hK2 on a “natural” substrate, namely PSA was examined. PSA was purified either from the prostates of BALB/c.PSA transgenic mice or tumor cell line supernatant (L1/PSA/IL2). hK2 was purified from tumor cell line supernatant (P815/hK2) and has been shown previously to be enzymatically active. The hK2 was purified using the hK2-specific antibody 6B7 and PSA was purified using the PSA-specific antibody RLS-DO6. The purified PSA was first tested for enzymatic activity when incubated with various antibodies, as shown in FIGS. 7A and 7B. This was done by pre-incubating PSA with various antibodies, followed by the addition of a chromogenic substrate for PSA (S-2586) and monitoring the change in absorbance over time. The PSA purified from the BALB/c.PSA prostates (FIG. 7A) shows a large amount of enzymatic activity, while there is a small amount of enzymatic activity of PSA purified from the cell culture supernatant (FIG. 7B). The activity does not change when incubated with 3C7, which is hK2-specific, or a mouse IgG1 isotype control. However, when PSA is incubated with a monoclonal antibody that is known to block PSA activity the enzymatic activity is completely blocked. This illustrates that most of the PSA purified from the prostates is active, while most of the PSA purified from the cell culture supernatant appears inactive. In order to see if hK2 would have an effect on the activity of PSA, hK2 was incubated with the “inactive” PSA, and monitored again for PSA activity. These results are shown in FIG. 7C. The activity of PSA is increased 500% when incubated with hK2, which is evident when PSA, hK2, and the IgG1 isotype control are mixed. Since hK2 cannot cleave the S-2586 substrate (data not shown), this increase is due to hK2 further activating PSA. Interestingly, 3C7 incubated with PSA and hK2 inhibits the activity of PSA to near the baseline activity seen in FIG. 7B. Since 3C7 is hK2-specific, 3C7 must be inhibiting the activity of PSA by inhibiting the activity of hK2. This figure demonstrates that hK2 can activate PSA, and that inhibition of hK2 by 3C7 is also an efficient way to inhibit the activity of PSA.

The monoclonal antibodies 6B7 and 11C4 showed reactivity to AV12-hK2 supernatant, containing a recombinant form of hK2, confirming their reactivity with hK2. AV12-hK2 supernatant was a generous gift from Hybritech.

### 2. Example 2 Use of Tumor Cells to Present Antigens in vivo

Tumor cells transfected with cDNAs encoding non-self proteins were used to investigate the ability of the immune system to respond to immunogenic antigens expressed by tumors. Secreted, intracellular, and surface proteins were used as model antigens, as these reflect the potential forms of tumor antigens. Syngeneic BALB/c mice injected with viable line 1 lung carcinoma or EMT6 mammary tumor cells secreting ovalbumin (Ova) or prostate-specific antigen (PSA) produced very high IgG antibody titers, equivalent to those in mice injected with protein in complete Freund's adjuvant (CFA). Secretion of the antigens was not necessary as tumor cells expressing a cell surface antigen (HER-2/Neu) or an intracellular antigen, green fluorescence protein (GFP), also generated high titer antigen-specific IgG antibodies. In IL-4 deficient mice, both IgG1 and IgG2a were produced in response to ovalbumin in CFA, whereas in response to tumor produced antigen, the antibodies switched from predominantly IgG1 to IgG2a indicating that the mechanisms responsible for antibody induction differed between these forms of immunization. In contrast to the line 1 and EMT6 tumors, which are of BALB/c origin, Ova or PSA producing B16 melanoma cells, which are of C57BL/6 origin, failed to elicit antibody production. This was not due to strain differences as a similar finding was observed when the tumors were grown in (BALB/c×C57BL/6) F1 mice, but appeared to be due to intrinsic differences in the tumors. Further more, co-injection of both B16/Ova and line 1 tumors resulted in production of anti-Ova antibody indicating that B16 tumors were not immunosuppressive, but instead line 1 tumors appear to exert an adjuvant effect.

a) Materials and Methods

(1) Animals and Cell Lines:

BALB/cByJ (H-2^{d}), C57BL/6 (H-2^{b}), (BALB/cByJ×C57BL/6)F1 (H-2^{d/b}) and IL-4 deficient (BALB/c-Il4^{tm2Nnt}) mice were purchased from the Jackson Laboratory (Bar Harbor, Me.) and used at 2-4 months of age.

Line 1, a small cell lung carcinoma (Yuhas, J. M., et al., Cancer Res 1974; 34:722-8), and EMT6.8, a clone of EMT6 (Rockwell, S. C., et al., J Natl Cancer Inst 1972; 49:735-49), a mammary carcinoma, both arose spontaneously in BALB/c mice and have been described previously. The B16F0 cell line was obtained from ATCC and was first characterized as a spontaneously arising melanoma in C57BL/6 (H-2^{b}) mice (Fidler, I. J. Cancer Res 1975; 35:218-24). cDNAs encoding ovalbumin (Ova) and prostate specific antigen (PSA) were transfected into line 1 or EMT6 tumor cells using lipofection and positive clones were isolated by limiting dilution analysis as previously described (Pulaski, B. A., et al., Proc Natl Acad Sci USA 1996; 93:3669-74, Wei, C., et al., Cancer Immunol Immunother 1996; 42:362-8). Line 1 transfected with the cDNA for green fluorescent protein (GFP) was a generous gift from Dr. Sandra Gollnick(Roswell Park Cancer Institute). Other GFP constructs can be used to transfect the L1 cell line, such as that disclosed Cemazar M. et al. (Cemazar M. et al., Cancer Gene Therapy. 9(4):399-406, 2002) which is herein incorporated by reference at least for material related to GFP). EMT6 transfected with human HER-2/neu was kindly provided by Dr. Pia Challita-Eid University of Rochester). Other HER-2/neu vectors can also be used, such as that disclosed by Penichet et al. (Penichet M L. Et al., Cancer Immunology, Immunotherapy. 49(12):649-62, 2001) which is herein incoproated by reference at least for material related to HER-2/neu). Cell lines were tested routinely for the presence of mycoplasma using the Gen-Probe detection system (Gen-Probe, San Diego, Calif.). Only cell lines testing negative were used in experiments.

(2) Flow Cytometric Analysis of LN Cell Populations:

BALB/c mice were injected intramuscularly (i.m.) with 1×10⁵ line 1/Ova tumor cells and tumors were allowed to grow for 15-21 days. After leg diameters had reached 12-14 mm, iliac (tumor draining) and inguinal (non-tumor draining) lymph nodes (LN) were removed and dissociated into single cell suspensions. One×10⁶ LN cells were incubated with fluorescein-isothiocynate (FITC) conjugated antibodies to cell surface markers B220, CD4 and CD8 (clones RA3-6B2, RM4-5 and 53-6.72 respectively, Pharmingen, San Diego Calif.). FITC-conjugated rat IgG2a (Pharmingen) was used as a negative control. Samples were analyzed using an EPICS Elite flow cytometer (Coulter Corp., Hialeah, Fla.)

(3) Injection of Cell Lines and Serum Collection:

Levels of Ova and PSA secreted in the supernatant were analyzed by protein specific ELISA assays as previously described (Pulaski, B. A., et al., Proc Natl Acad Sci USA 1996; 93:3669-74). Levels of GFP and HER-2/neu protein in transfected tumor cells were determined by flow cytometric analysis (Table 1). Transfected tumors were injected i.m. into the left hind flank of BALB/c, C57BL/6, (BALB/c×C57BL/6)F1 or IL-4 deficient mice at cell numbers that produced tumors within 20 days. Experiments were halted at the end of the 3 week period or when the mean thigh diameter of the mouse was approximately 12-14 mm. Every 3-4 days during tumor growth, blood was collected, allowed to clot and the serum removed. In control experiments, 50 μg of Ova was emulsified in complete Freund's adjuvant (CFA, Sigma, St. Louis, Mo.) or mixed with Alhydrogel aluminum hydroxide (Accurate Chemical, Westbury, N.Y.) and injected i. m. For mixing experiments, 2×10³ parental line 1 cells were mixed with 2×10⁵ B16/Ova cells and injected i. m. into (BALB/c×C57BL/6) F1 mice. The cell numbers in these injections were selected to control for the different rates of in vivo growth between the two types of tumors. After 18-21 days mice were bled and tumors removed to assay for Ova expression. To determine the amount of Ova protein being produced directly ex vivo, tumors were collagenased to form single cell suspensions and 1×10⁷ cells were lysed in 1 ml NP-40 lysis buffer. Tumor lysates were analyzed by ELISA specific for Ova as previously described (Pulaski, B. A., et al., Proc Natl Acad Sci USA 1996; 93:3669-74). In the converse experiment, 2×10⁵ parental B16 cells were co-injected with 5×10⁴ line 1/Ova cells and allowed to grow for 18-21 days. Guidelines for the humane treatment of animals were followed as approved by the University Comnmittee on Animal Resources.

(4) ELISAs for the Detection of Mouse Antibodies:

Ova (Sigma), GFP (Clontech, San Diego, Calif.) or the extracellular domain of HER-2/neu (Genentech, San Francisco, Calif.) was coated onto ELISA plates at a concentration of 1 μg/ml in coating buffer (0.05 M boric acid in PBS, pH 9.5). Plates were blocked with PBS containing 2% FBS, 5 mM HEPES and 0.05% sodium azide. Dilutions of serum were prepared in the same buffer and were allowed to bind to the plate. Mouse immunoglobulin was detected with alkaline phosphatase conjugated goat anti-mouse second step reagents specific for IgM, total IgG, or IgG subtypes; IgG1 or IgG2a (Southern Biotechnology, Birmingham Ala.). Alkaline phosphatase was detected with p-nitrophenylphosphate (Calbiochem, San Diego, Calif.) in diethanolamine buffer (Sigma) and colorimetric changes were read at a wavelength of 405 nm. An assay specific for anti-PSA antibodies was developed as a sandwich ELISA in which the plate was coated with 3 μg/ml of polyclonal rabbit anti-human PSA (DAKO, Carpinteria, Calif.), blocked with PBS containing 5% powdered non-fat milk and 0.2% Tween-20 (blotto-tween), and then PSA (Calbiochem) antigen added at 30 ng/ml, followed by dilutions of the serum in blotto-tween. Bound antibodies were detected with HRP conjugated rabbit anti-mouse IgG (Jackson Immunoresearch, West Grove, Pa.) and developed using O-phenylenediamine (OPD, Sigma) as the chromagen. The optical density at 490 nm was determined using an ELISA reader. Results are expressed as endpoint dilutions where the endpoint is the final dilution which gives an optical density reading of twice background levels. This was done to normalize for the different ELISAs performed and the background level was determined using a negative control serum sample at a dilution of 1:20 from mice injected with untransfected parental tumor cells. Comparisons between groups were performed with a non-parametric test (Mann-Whitney U test).

b) Results

(1) Tumor Bearing Mice Have an Increased Percentage of B220⁺ Cells in the Tumor Draining Lymph Node:

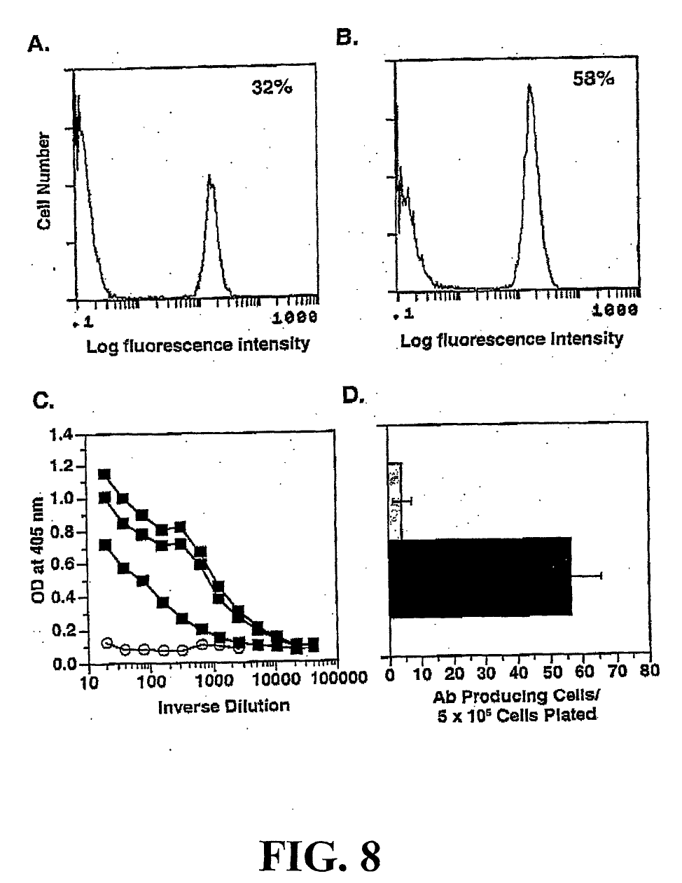

To investigate how tumors may promote or prevent immune responses, a poorly immunogenic mouse lung carcinoma, line 1 was chosen initially for study. The line 1 tumor cell line was transfected with cDNA encoding Ova to investigate the immune system's ability to respond to potentially immunogenic antigens expressed by tumors. Initial observations suggesting immune effectors may be expanding in response to line 1/Ova tumors where the enlargement and increased cellularity of the LN draining the tumor site. To further characterize these changes, the iliac (tumor draining) LN and inguinal (non-tumor draining) LN were removed from mice bearing line 1/Ova tumors. The total number of cells in the tumor draining LN increased 4-5-fold. The range of cell numbers was 1.2-3.4×10⁶ cells/LN in the non tumor draining LN with an average±SEM of 1.9±0.3×10⁶ cells/LN. The tumor draining LN, in contrast, had an average of 1.1±0.1×10⁷ cells/LN with a range of 7.5-15×10⁶ cells/LN. Flow cytometric analysis revealed that the percentage of B220⁺ cells in the tumor draining LN increased 2 fold over that of the non draining LN (Compare 58% to 32% in FIGS. 8A and 8B). The percentage of B220⁺ cells in the non draining LN was comparable to the percentage of B220⁺ cells in a naive LN. This expanded percentage of B cells and the increased overall cellularity resulted in a 7-8-fold increase in B cells in the tumor draining LN. There was a corresponding decrease in the percentage of CD4⁺ and CD8⁺ cells in these LN. However, when the expansion in total cell number was taken into account, there was a 2-fold increase in absolute numbers of CD4⁺ and CD8⁺ T cells demonstrating that while both T and B cells increased in number, the B cells were preferentially expanded.

This marked increase in the B cell population suggested that B cells had been activated in response to the growing line 1/Ova tumors. To examine this possibility, serum was collected from tumor bearing mice and assayed in an ELISA specific for anti-Ova antibodies. FIG. 8C shows a representative ELISA of sera from 3 individual mice injected with line 1/Ova tumors. Each of the 3 mice showed high levels of anti-Ova IgG antibodies whereas serum from a representative mouse injected with parental line 1 cells did not contain Ova specific antibodies. Endpoint titers in these mice fell within the range of 1,000-10,000 suggesting that a vigorous B cell response had been activated in response to the Ova secreting tumors. Tumor draining LN cells were also analyzed for Ova specific antibody producing cells by ELISPOT assays. This assay has the advantage of determining the actual number of antibody-producing B cells present, rather than just the quantity of antibody made. Consistent with the serum analysis, this assay demonstrated that the frequency of Ova specific B cells was markedly increased in the tumor draining LN (FIG. 8D).

(2) Line 1/Ova Tumors Elicit High Anti-Ova Antibody Titers Comparable to Ova Emulsified in Adjuvant:

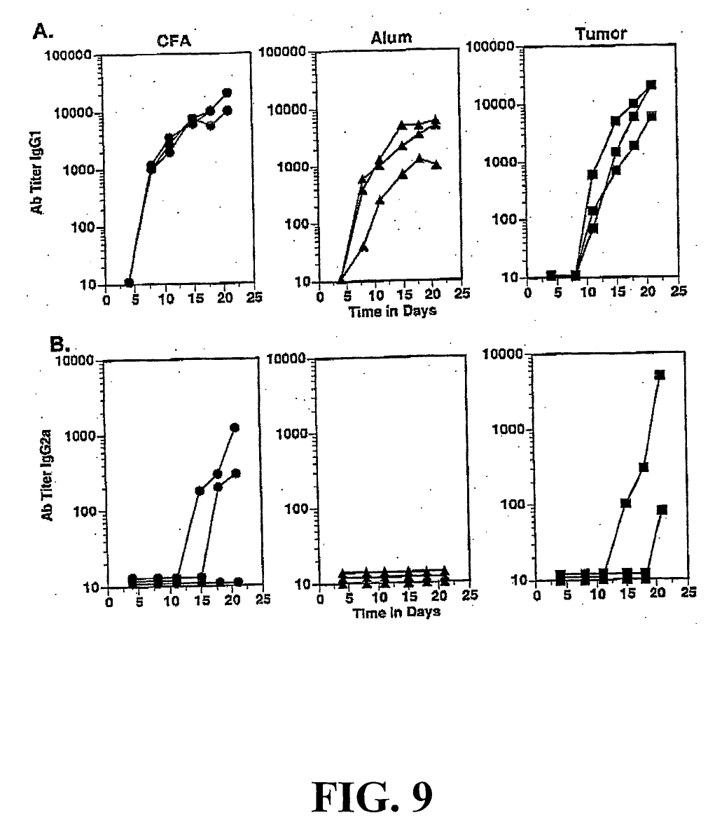

To further investigate the B cell response in tumor bearing mice, the isotype class of the IgG antibody responses in line 1/Ova injected mice was compared with that in mice immunized with Ova emulsified in CFA or mixed with aluminum hydroxide (alum). CFA was used because of its high potency and its induction of IgG1 antibody secretion early in the primary response with eventual switching to IgG2a, a profile consistent with a predominant Th1 T helper cell response (Grun, J. L. and Maurer, P. H., Cell Immunol 1989; 121:134-145). In contrast, alum induces IgG1 secretion and little or no IgG2a, an isotype pattern characteristic of Th2 type immunity (Grun, J. L. and Maurer, P. H., Cell Immunmol 1989; 121:134-145). These two adjuvants were used to compare the kinetics, magnitude and qualitative nature of the immune response elicited by progressively growing line 1/Ova tumors to that obtained with well-characterized adjuvants. It is not possible to accurately determine the amount of Ova antigen delivered by the tumors over time, but based on the amount of antigen produced by the cells in vitro (Table 3) and the growth kinetics of the tumor, it was estimated that about 50 μg of Ova would be an equivalent or higher dose so this amount was used with the adjuvants. FIG. 9A shows the IgG1 antibody titer in mice injected with Ova in CFA or in alum or with line 1/Ova cells as a function of time. Sera from mice were collected every 3-4 days beginning 4 days post injection and continuing until 21 days after injection. As expected, mice injected with Ova in CFA or alum produced high titer anti-Ova IgG1 antibodies, which were detectable early after immunization and increased throughout the duration of the experiment. Surprisingly, mice given a single injection of viable line 1/Ova tumor cells produced high anti-Ova IgG1 antibody responses similar to those seen in mice injected with Ova emulsified in CFA. The kinetics of the antibody response in tumor bearing mice were slightly delayed; anti-Ova IgG1 antibodies were not detected until days 11-15. Nevertheless, the end point titers at day 21 in tumor bearing mice were as high as in mice injected with Ova in CFA (geometric mean titers were approximately 13000 and 15000 respectively),. Anti-Ova IgG2a antibody titers were also examined in tumor bearing mice and again compared to responses induced with antigen in adjuvant (FIG. 9B). Similar to previously published results, Ova mixed in alum did not induce IgG2a synthesis at any time point tested (Grun, J. L. and Maurer, P. H., Cell Immunol 1989; 121:134-145). Two out of 3 mice injected with Ova in CFA produced anti-Ova IgG2a antibodies by day 15 after injection. With approximately similar kinetics, 2 out of 3 mice injected with line 1/Ova tumors also produced anti-Ova IgG2a antibodies. Therefore, the antibody response induced by Ova expressing line 1 tumor cells appears to be similar to Ova in CFA and indeed, by this criterion, more potent than alum. In addition, the pattern of isotype switching was similar between the tumor and CFA immunized mice.

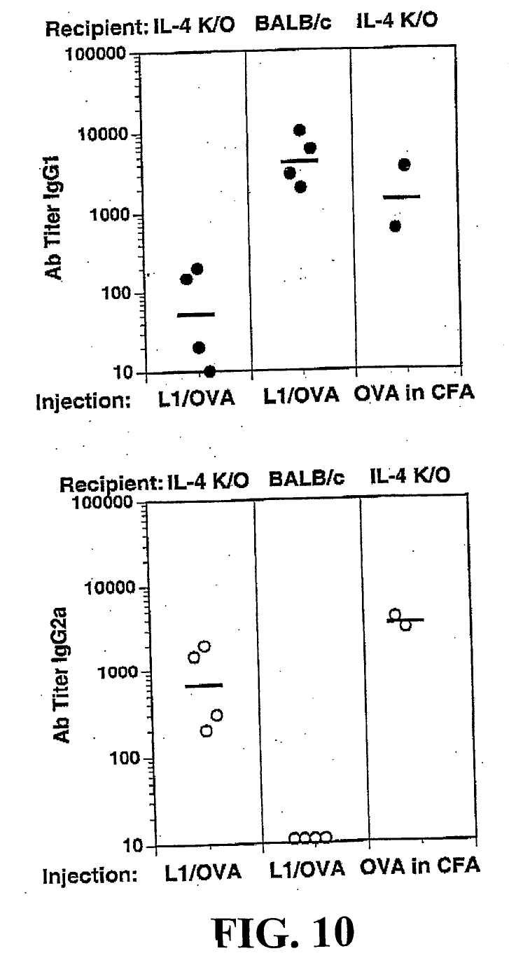

To further investigate the mechanisms involved in the generation of these high titer anti-Ova antibodies, the antibody responses in IL-4 deficient mice were examined. This cytokine plays a major role in the production of antibodies of the IgG1 isotype (Snapper, C. M., et al., Immunol Rev 1988; 102:51-75). Thus it was determined if IL-4 might be essential for the induction of anti-Ova antibodies. Either line 1/Ova cells or Ova in CFA was injected into IL-4 knockout mice, sera collected and analyzed for the presence of anti-Ova antibodies of both the IgG1 and IgG2a subtypes. The results clearly demonstrate that IL-4 is not essential for eliciting an IgG antibody response when mice are immunized with Ova expressed by tumor cells or emulsified in CFA (FIG. 10). Interestingly, immunization of IL-4 deficient mice with tumor cells, although still eliciting a response, caused a marked change in the isotype of the antibodies produced. In this experiment control BALB/c mice generated antibodies exclusively of the IgG1 isotype with titers similar to what was observed in earlier experiments (see FIG. 9) while IL-4 deficient mice produced only low titers of anti-Ova IgG1. At day 18 post tumor inoculation, control BALB/c mice did not produce anti-Ova IgG2a in this experiment however, there was a marked shift to production of IgG2a antibodies in IL-4 deficient mice. Immunization of the IL-4 knock-out mice with Ova in CFA also resulted in high titers of IgG2a antibodies, but in contrast to the mice immunized with the line 1/Ova, high titers of IgG1 antibodies were also maintained. These results show that immunization with Ova expressing tumor cells can elicit high titer antigen specific IgG antibodies in the absence of IL-4, however, the isotype of the antibodies elicited has switched from predominantly IgG1 in normal BALB/c to IgG2a in the IL-4 deficient mice. In contrast, IL-4 has less of an impact on antibody production when mice are immunized with Ova emulsified in CFA in that both IgG1 and IgG2a responses are maintained with high titers.

(3) Transfection of BALB/c Derived Tumors with Different Antigens can Induce Antigen Specific High Titer IgG Antibodies.

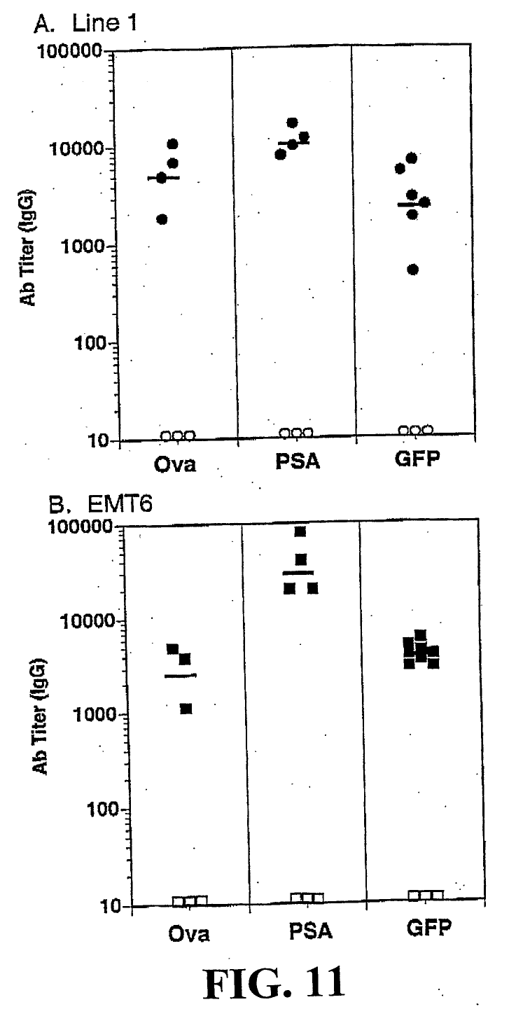

The above results demonstrated that line 1/Ova tumors could induce high titer anti-Ova antibodies and class switching to IgG isotypes. Surprisingly, these responses were equal to and sometimes surpassed those seen in mice given an injection of Ova in adjuvants. To determine if this response was unique to Ova, a strong antigen used in many experimental systems, line 1 tumors were also transfected with cDNAs encoding PSA or GFP. Although PSA and Ova are secreted proteins, GFP is an intracellular protein. Use of this antigen allowed determination of whether the protein produced by the tumor cells needs to be secreted for a response to be elicited. BALB/c mice were injected with various concentrations of tumor cell lines and antigen specific total IgG was assayed by ELISA as described in the Materials and Methods. FIG. 11A shows the individual responses and-geometric mean titers of mice injected with line 1 cells producing either Ova, PSA or GFP. In all mice tested, Ova expressing line 1 tumors induced high anti-Ova antibody titers similar to the results seen in FIGS. 8 and 9. Similarly, line 1 tumors expressing PSA induced strong anti-PSA antibody responses. Perhaps more surprisingly, line 1/GFP tumor cells also generated high titer IgG anti-GFP antibodies. Since T cell help is required to promote B cell isotype switching to IgG, this result suggests that intracellular tumor antigens can induce both B cell and T cell responses.

To determine if the effect is unique to line 1 tumors, similar experiments using another tumor model, EMT6, a spontaneously arising mammary carcinoma, were performed. As shown in Table 3, these cells secrete slightly less Ova than the line 1/Ova cell line. Additionally, to control for the secreted nature of the Ova antigen, EMT6 cells were transfected with cDNA for HER-2/neu, a cell surface protein and used to determine if mice could generate antibodies to surface determinants. Transfected EMT6 tumors also induced high titer antigen specific IgG antibodies (FIG. 11B). Interestingly, even though the amount of Ova secreted by EMT6 cells is lower than the line 1 cell line, the geometric mean titer in response to EMT6/Ova tumors was as high as that observed in response to line 1/Ova tumors. Similarly, the level of human HER-2/neu on the surface of EMT6 cells appears to be low (Table 3), yet high titer anti-HER-2/neu antibodies were induced. The mean geometric titers of serum from mice injected with antigen expressing EMT6 cells were between 3000 and 5000. In summary, in two different tumor models, line 1 and EMT6, it was possible to generate strong antibody responses to secreted proteins (Ova, PSA), to an intracellular protein (GFP) and to a cell surface protein (HER-2/neu).

(4) Antigen Expressing B16 Melanoma Cells do not Induce Antigen Specific IgG Antibodies in C57BL/6 or (BALB/c×C57BL/6)F1 Mice.

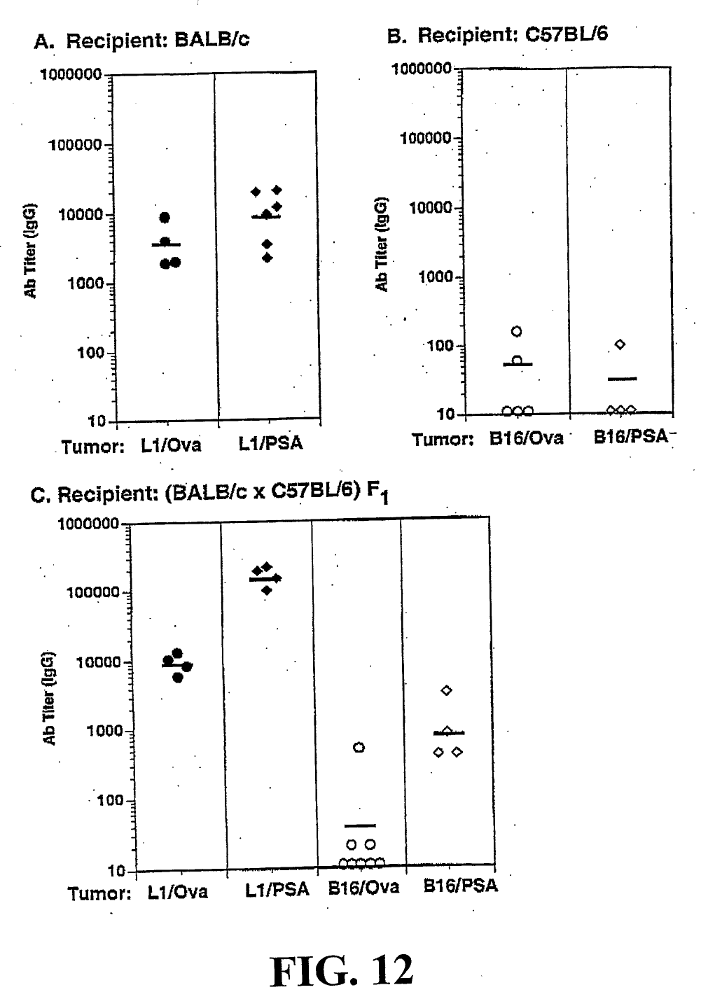

Line 1 and EMT6 cells are derived from the same strain of mouse, BALB/c. Immune responses can be significantly influenced by both MHC and background genes. It was therefore of interest to determine if these results were similar in a different strain and tumor model. The B16 melanoma cell line was employed, and as described above, transfected with cDNA encoding either Ova or PSA. Since B16 is of H-2^{b} origin, C57BL/6 mice were injected with either B16/Ova or B16/PSA tumor cells. As in previous experiments mice were bled between 16 and 21 days post tumor implantation, and antigen specific antibodies assayed by ELISA. For comparison, sera from BALB/c mice with growing line 1/Ova tumors were used as controls. FIG. 12A again demonstrates that line 1 tumors induced antigen specific antibodies in BALB/c mice in all animals tested, however, FIG. 12B shows that B16 tumors did not elicit a strong antibody response in C57BL/6 mice. For example, only two out of 5 mice injected with B16/Ova showed detectable levels of anti-Ova antibodies (FIG. 12B). Similarly, only one of the mice injected with B16/PSA tumors had detectable levels of anti-PSA IgG (FIG. 12B).

The different results obtained using B16 in its syngeneic host C57BL/6 compared to those obtained with tumors in BALB/c mice, could be due to strain differences, as have been well-documented in other experimental systems. For example, in the mouse model of leishmaniasis, Leishmania major infection of BALB/c mice promotes a Th2 type response characterized by high antibody titers, but the mice are not protected. In contrast, infection of C57BL/6 mice induces effective immunity characterized by a Th1 type response and clearance of the parasite (Reiner, S. L. and Locksley, R. M., Annu Rev Immunol 1995; 13:151-77). To determine if strain differences were contributing to the contrasting antibody responses observed above, an experiment was performed in which line 1/Ova or B16/Ova cells were injected into (BALB/c×C57BL/6)Fl mice. If strain differences are crucial in determining the antibody response to tumors, then F1 mice would be expected to display the responder phenotype to both line 1 and B16 tumors. FIG. 12C shows antigen specific antibody titers in F1 mice injected with either line 1 or B16 tumor cells. F1 mice injected with line 1/Ova or with line 1/PSA tumor cells generated high antibody titers, comparable to or higher than those seen in BALB/c mice. In contrast, B16/Ova injected F1 mice generated weak or nonexistent antibody responses to Ova, similar to that found in C57BL/6 mice. Whereas the B16/PSA challenged F1 mice exhibited higher titers of anti-PSA antibodies than the same tumors in C57BL/6 mice, they were significantly lower than the response to PSA produced by line 1 cells. This suggests a relatively modest strain effect. Thus, while there is evidence for a strain effect of the recipient (compare the response to B16/PSA in C57BL/6 to that in the F1 mice), these experiments indicate that the tumor line itself contributes substantially to the magnitude of the antibody response.

(5) Line 1 Tumors Act as Adjuvants to induce Anti-Ova Antibodies in Response to B16/Ova Tumors.

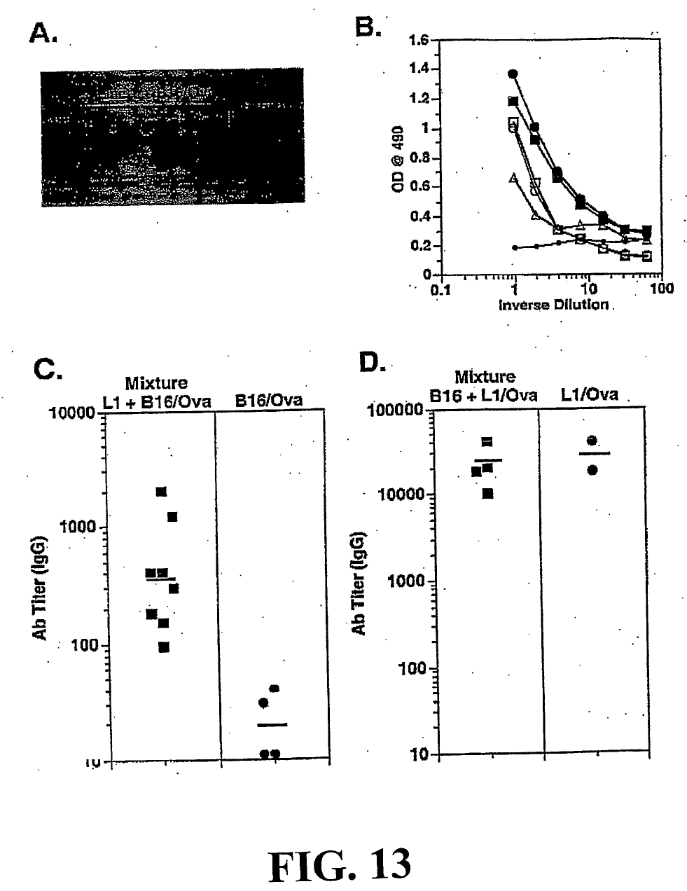

The apparent discrepancy in response to antigens expressed by different tumors could be explained by at least two distinct mechanisms. Line 1 cells could produce factors that enhance the immune response (act as an adjuvant) or alternatively, B16 cells could produce factors that suppress a response which normally would occur. To address these possibilities, B16/Ova cells were mixed with parental line 1 cells and antibody synthesis in (BALB/c×C57BL/6)F1 mice was analyzed. F1 mice were used in these experiments to allow the simultaneous growth of B16/Ova (H-2^{b}) and line 1 (H-2^{d}) tumors. In this protocol, the antigen is delivered by the B16 tumor and the potential adjuvant effect by the line 1 tumor. Mice were bled between days 18 and 21 and the serum assayed for anti-Ova IgG antibodies. At sacrifice, the tumors were examined to determine that both cell lines had indeed grown. Due to its melanin production, growth of the B16/Ova melanoma is readily distinguishable from the nonpigmented line 1 tumor, and at the time of sacrifice it was clear from the interspersed areas of pigmented and nonpigmented tissue that both tumor types had grown (FIG. 13A). Additionally, F1 mice were injected with B16/Ova tumors alone as a negative control. Only pigmented tissue was visible in these B16/Ova tumors (FIG. 13A). To confirm that B16/Ova cells were indeed growing and antigen was still being expressed, the amount of Ova protein expressed by B16/Ova cells injected alone and in combination with parental line 1 cells was analyzed by ELISA (FIG. 13B). The amount of Ova expressed by B16/Ova tumors growing in vivo was not significantly different than the B16/Ova cells growing in vitro, suggesting that a negative variant of B16/Ova did not selectively outgrow from the original tumor (FIG. 13B). Furthermore, tumors excised from mice given a combination of line 1 and B16/Ova cells continued to express Ova at the end of the experiment. The levels of Ova in these samples were slightly lower than in the B16/Ova tumor alone due to the concomitant growth of line 1 interspersed with B16/Ova cells (FIG. 13A and 13B). FIG. 13C demonstrates that injection of line 1 cells in conjunction with B16/Ova induced a significant response compared to B16/Ova tumors alone. The response generated to the mixed tumor is somewhat lower than to line 1/Ova alone (compare FIG. 13C to FIG. 12C). However, the co-administration of line 1 and B16/Ova clearly had a positive effect over that of B16/Ova given alone. To determine if the B16 parental tumor could exert any suppressive effect on antibody synthesis in response to line 1/Ova tumors, F1 mice were injected with either line 1/Ova alone (as in FIG. 12C) or a combination of line 1/Ova and parental B16 tumors. In contrast to the experiment in FIG. 13C, the antigen is now being delivered by the line 1 tumor in order to assess any suppressive effect contributed by the B16 tumor. FIG. 13D demonstrates that B16 tumors have no suppressive effect on anti-Ova antibody synthesis in F1 mice and the titers in mice injected with both line 1/Ova mixed with B16 are as high as in mice injected with line 1/Ova alone. Therefore, the adjuvant effect of line 1 tumors to promote antibody synthesis is dominant over any suppressive effect that the B16 tumors may have in inhibiting the induction of antibody synthesis.

TABLE 3
Amounts of antigen produced by various tumor transfectants.

| Tumor Cell | Haploty | Ova | PSA | GFP | HER-2/neu |
|---|---|---|---|---|---|
| Line* | pe | ng/ml† | ng/ml† | (MFI)‡ | (MFI) |
| Line 1 | H-2^{d} | 25 | 8 | 165 | N/A |
| EMT6 | H-2^{d} | 10 | 45 | N/A | 0.362 |
| B16 | H-2^{b} | 5 | 16 | N/A | N/A |
| *Each tumor cell line represents a cell line transfected with a single antigen. In all, eight individual transfectants were utilized in this study. | | | | | |
| †Protein levels were assayed by ELISA from culture supernatant. Culture supernatants were collected form 2 × 10⁵ cells incubated for 48 hr in 2 ml media. | | | | | |
| ‡Line 1/GFP tumor cells were assayed for levels of intracellular GFP by flow cytometric analysis and | | | | | |
| # values represent mean fluorescent intensity (MFI). | | | | | |
| EMT6/HER-2/neu cells were stained with mouse anti-human HER-2/neu followed by | | | | | |
| # incubation with goat anti-mouse FITC and subjected to flow cytometric analysis. | | | | | |

1. A composition for generating an antibody response to an antigen, comprising a cell, wherein the cell comprises a vector that encodes the antigen.

2. The composition of claim 1, wherein the cell is derived from a cell line.

3. The composition of claim 2, wherein the cell line is a tumor cell line.

4. The composition of claim 3, wherein the tumor cell line is L1, EMT6, or CT-26.

5. The composition of claim 1, wherein the antigen comprises hK2

6. The composition of claim 1, wherein the antigen is secreted.

7. The composition of claim 1, wherein the antigen is bound to the cell surface.

8. The composition of claim 1, wherein the antigen is internal to the cell.

9. The composition of claim 1, wherein the antigen comprises a peptide or protein produced by a virus.

10. The composition of claim 9, wherein the virus is a Herpes simplex virus type-1, Herpes simplex virus type-2, Cytomegalovirus virus, Epstein-Barr virus, Varicella-zoster virus, Human herpesvirus 6 virus, Human herpes virus 7, Human herpes virus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, or Human Immunodeficiency virus type-2.

11. The composition of claim 1, wherein the antigen comprises a peptide or protein produced by a bacterium.

12. The composition of claim 11, wherein the bacterium is a Mycobacterium, Nocardia, Legionella, Salmonella, Shigella, Yersinia, Pasteurella, Actinobacillus, Listeria, Brucella, Cowdria, Chlamydia, Coxiella, Rickettsial, Ehrlichia, Staphylococcus, Streptococcus, Bacillus, Escherichia, Vibrio, Campylobacter, Neiserria, Pseudomonas, Haemophilus, Clostridium, Yersinia.

13. The composition of claim 11, wherein the bacterium is Mycobacterium tuberculosis, Mycobacterium bovis, Mycobacterium bovis strain BCG, Mycobacterium avium, Mycobacterium intracellulare, Mycobacterium africanum, Mycobacterium kansasii, Mycobacterium marinum, Mycobacterium ulcerans, Mycobacterium avium, paratuberculosis, Nocardia asteroides, Legionella pneumophila, Salmonella typhi, Shigella, Yersinia pestis, Pasteurella haemolytica, Pasteurella multocida, Actinobacillus pleuropneumoniae, Listeria monocytogenes, Listeria ivanovii, Brucella abortus, Cowdria ruminantium, Chlamydia pneumoniae, Chlamydia trachomatis, Chlamydia psittaci, Coxiella burnetti, Rickettsial, Ehrlichia, Staphylococcus aureus, Staphylococcus epidermidis, Streptococcus pyogenes, Streptococcus agalactiae, Bacillus anthracis, Escherichia coli, Vibrio cholerae, Campylobacter, Neiserria meningitidis, Neiserria gonorrhea, Pseudomonas aeruginosa, Haemophilus influenzae, Haemophilus ducreyi, Clostridium tetani, Yersinia enterolitica.

14. The composition of claim 1, wherein the antigen is a protein or peptide produced by a parasite.

15. The composition of claim 14, wherein the parasite is a Toxoplasma, Plasmodium, Trypanosoma, Leishmania, Schistosoma, or Entamoeba.

16. The composition of claim 14, wherein the parasite is a Toxoplasma gondii, Plasmodium falciparum, Plasmodium vivax, Plasmodium malariae, Trypanosoma brucei, Trypanosoma cruzi, Leishmania major, Schistosoma mansoni, other Schistosoma, Entamoeba histolytica.

17. The composition of claim 1, wherein the antigen is a protein or peptide produced by a cancer cell.

18. The composition of claim 17, wherein the cancer cell is a Hodgkins lymphoma cell, non-Hodgkins lymphoma cell, B cell lymphoma cell, T cell lymphoma cell, myeloid leukemia cell, leukemias cell, mycosis fungoides cell, carcinoma cell, squamous cell carcinoma cell, adenocarcinoma cell, sarcoma cell, glioma cell, blastoma cell, neuroblastoma cell, plasmacytoma cell, histiocytoma cell, melanoma cell, adenoma cell, hypoxic tumour cell, myeloma cell, AIDS-related lymphoma cell, AIDS related sarcoma cell, metastatic cancer cell, bladder cancer cell, brain cancer cell, nervous system cancer cell, squamous cell carcinoma cell, neuroblastoma cell, glioblastoma cell, ovarian cancer cell, skin cancer cell, liver cancer cell, melanoma cell, colon cancer cell, cervical cancer cell, cervical carcinoma cell, breast cancer cell, epithelial cancer cell, renal cancer cell, genitourinary cancer cell, pulmonary cancer cell, esophageal carcinoma cell, hematopoietic cancer cell, testicular cancer cell, colo-rectal cancer cell, prostate cancer cell, or pancreatic cancer cell.

19. A composition comprising a substance, wherein the substance preferentially binds hK2 over PSA.

20-27 (canceled)

28. A method of making an antibody comprising injecting an antigen expressing cell into an animal such that the animal has an immune response and isolating antibodies produced by the immune response.

29-59 cancelled

60. A method of screening a sample for the presence of hK2 comprising contacting the sample with a composition that preferentially binds hK2 over PSA.

61-75 cancelled

76. A method of modulating hK2 activity comprising contacting a sample with tion that modulates hK2 activity.

77-101 cancelled

102. A method of treating a subject with a cancer comprising administering to the subject a composition that preferentially binds hK2 over PSA.
